(19) United States
(12) Patent Application Publication
(10) Pub. No.: US 2021/0161958 A1
(43) Pub. Date: Jun. 3, 2021

(54) COMPOSITIONS AND METHODS FOR TREATING MELANOMA WITH A CHIMERIC ANTIGEN RECEPTOR

(71) Applicant: THE TRUSTEES OF THE UNIVERSITY OF PENNSYLVANIA, Philadelphia, PA (US)

(72) Inventors: Todd Ridky, Bryn Mawr, PA (US); Christopher Natale, Philadelphia, PA (US); Aimee S. Payne, Merion Station, PA (US); Christoph T. Ellebrecht, Philadelphia, PA (US)

(21) Appl. No.: 17/047,993

(22) PCT Filed: Apr. 17, 2019
(86) PCT No.: PCT/US2019/027943

§ 371 Date: Oct. 15, 2020

Related U.S. Application Data
(60) Provisional application No. 62/660,037, filed on Apr. 19, 2018.

(51) Int. Cl. A61K 35/17; C07K 14/72; C07K 14/705; C07K 14/725; C07K 16/28

(52) U.S. Cl. CPC A61K 35/17; C07K 14/72; C07K 14/70517; A61K 38/00; C07K 16/2818; C07K 16/2869; C07K 14/7051

(57) ABSTRACT

The present invention includes compositions and methods for using a chimeric antigen receptor that specifically binds to melanocortin receptor (MCR). The present invention relates generally to the treatment of a patient having cancer that expresses one or more melanocortin receptors (MCR), such as melanoma. The invention also includes methods of making a genetically modified T cell expressing a chimeric antigen receptors (CAR) that binds to MCR.

### CROSS-REFERENCE TO RELATED APPLICATION

The present application is entitled to priority under 35 U.S.C. § 119(e) to U.S. Provisional Patent Application No. 62/660,037 filed Apr. 19, 2018, which is hereby incorporated by reference in its entirety herein.

### STATEMENT REGARDING FEDERALLY SPONSORED RESEARCH OR DEVELOPMENT

This invention was made with government support under Grant No. R01-AR068288 awarded by the National Institutes of Health. The government has certain rights in the invention.

### BACKGROUND OF THE INVENTION

Invasive melanoma is the most lethal and costly skin cancer with over 75,000 new cases and 10,000 deaths per year in the U.S. In 2011, the cost to treat these patients was estimated at $5 billion. This figure does not include new biologic immunotherapeutics available over the past 3 years which increase survival, but cost between $300K-$1M per year to treat one patient. Although these immune checkpoint inhibitor blocking antibodies targeting PD-1 and CTLA-4 lead to long-term survival in about 30% of patients, most with metastatic disease still succumb to their tumor.

Immunotherapy is a promising approach for cancer treatment thanks to the potential of the immune system to target tumors without the toxicity associated with traditional chemo-radiation.

However, there is an urgent need for a more targeted antigen-specific immunotherapy for treatment of skin cancers, such as, for example, melanoma. The present invention addresses this need.

### SUMMARY OF THE INVENTION

Provided is an isolated nucleic acid sequence encoding a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof. In some embodiments, the MCR binding domain comprises at least one MCR peptide ligand selected from the group consisting of: an alpha-melanocyte stimulating hormone (aMSH), an Agouti protein and any mutant or variant thereof. In some embodiments, the MCR binding domain is encoded by a nucleic acid sequence selected from the group consisting of SEQ ID NOs: 5, 7 and 9. In some embodiments, the transmembrane domain comprises a CD8 alpha hinge and transmembrane domain. In further embodiments, the signaling domain comprises a CD3 signaling domain. In further embodiments, the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof. In yet further embodiments, the CAR is encoded by a nucleic acid sequence selected from the group consisting of SEQ ID NOs: 11, 12, 15, 16, 19, and 20. In yet further embodiments, the MCR binding domain specifically binds to MCR expressed on tumor cells.

Also provided is a vector comprising the isolated nucleic acid sequence of any one of the previous embodiments.

Provided is an isolated chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof. In some embodiments, the MCR binding domain comprises at least one MCR peptide ligand selected from the group consisting of: an alpha-melanocyte stimulating hormone (aMSH), an Agouti protein and any mutant or variant thereof. In some embodiments, the MCR binding domain comprises an amino acid sequence selected from the group consisting of SEQ ID NOs: 1-4, 6, 8 and 10. In some embodiments, the transmembrane domain comprises a CD8 alpha hinge and transmembrane domain. In further embodiments, the signaling domain comprises a CD3 signaling domain. In further embodiments, the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof. In yet further embodiments, the MCR binding domain specifically binds to MCR expressed on tumor cells. In yet further embodiments, the CAR comprises an amino acid sequence selected from the group consisting of SEQ ID NOs: 13, 14, 17, 18, 21 and 22.

Also provided is a cell comprising the nucleic acid sequence or the isolated CAR of any one of the previous embodiments.

Provided is a modified cell comprising a nucleic acid sequence encoding a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof. In some embodiments, the MCR binding domain specifically binds to MCR expressed on tumor cells. In some embodiments, the tumor cells are from melanoma. In some embodiments, the cell is selected from the group consisting of a T cell, a natural killer (NK) cell, a cytotoxic T lymphocyte (CTL), a regulatory T cell and a macrophage.

Also provided is a composition comprising the modified cell of any one of the previous embodiments.

Provided is the use of the cell of any one of the previous embodiments in the manufacture of a medicament for the treatment of a cancer or a disease, disorder and condition associated with dysregulated expression of MCR in a subject in need thereof.

Provided is a method for stimulating a T cell-mediated immune response to a melanocyte cell population in a subject, the method comprising administering to the subject an effective amount of a modified cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof.

Provided is a method of treating a subject with a cancer or a disease, disorder and condition associated with dysregulated expression of MCR, the method comprising administering to the subject a modified T cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof. In some embodiments, the cancer is melanoma. In some embodiments, the modified T cell is autologous to the subject. In some embodiments, the method further comprises administering to the subject an additional agent selected from the group consisting of a chemotherapeutic agent, an anti-cell proliferation agent, an immunotherapeutic agent, an antitumor vaccine and any combination thereof. In further embodiments, the modified T cell and the additional agent are co-administered to the subject. In yet further embodiments, the additional agent is an anti-programmed cell death 1 (PD-1) antibody.

Provided is an isolated nucleic acid sequence encoding a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In some embodiments, the MCR binding domain comprises a heavy and light chain. In some embodiments, the MCR binding domain is selected from the group consisting of a human antibody, a humanized antibody, and a fragment thereof. In further embodiments, the antibody or a fragment thereof is selected from the group consisting of a Fab fragment, a F(ab′)₂ fragment, a Fv fragment, and a single chain Fv (scFv). In some embodiments, the MCR binding domain specifically binds to MCR expressed on tumor cells. In some embodiments, the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

Provided is a vector comprising the isolated nucleic acid sequence of any one of the previous embodiments.

Also provided is an isolated chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In some embodiments, the MCR binding domain comprises a heavy and a light chain. In some embodiments, the MCR binding domain is an antibody selected from the group consisting of a human antibody, humanized antibody, and fragment thereof. In some embodiments, the MCR binding domain is selected from the group consisting of a Fab fragment, a F(ab′)₂ fragment, a Fv fragment, and a single chain Fv (scFv). In some embodiments, the MCR binding domain specifically binds to MCR expressed by tumor cells. In some embodiments, the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

Provided is a cell comprising the isolated nucleic acid sequence or the isolated CAR of any one of the previous embodiments.

Also provided is a modified cell comprising a nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In some embodiments, the MCR binding domain specifically binds to MCR expressed by tumor cells. In some embodiments, the tumor cells are melanoma. In some embodiments, the cell is selected from the group consisting of a T cell, a natural killer (NK) cell, a cytotoxic T lymphocyte (CTL), a regulatory T cell and a macrophage.

Provided is a composition comprising the modified cell of any one of the previous embodiments.

Provided is use of the cell of any one of the previous embodiments in the manufacture of a medicament for the treatment of a cancer or a disease, disorder or condition associated with dysregulated expression of MCR in a subject in need thereof.

Provided is a method for stimulating a T cell-mediated immune response to a melanocyte cell population in a subject, the method comprising administering to the subject an effective amount of a modified cell that expresses a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

Also provided is a method of treating a subject with a cancer or a disease, disorder and condition associated with dysregulated expression of MCR, the method comprising administering to the subject a modified T cell that expresses a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In some embodiments, the cancer is a melanoma. In some embodiments, the modified T cell is autologous to the subject. In some embodiments, the method further comprises administering to the subject an additional agent selected from the group consisting of a chemotherapeutic agent, an anti-cell proliferation agent, an immunotherapeutic agent, an antitumor vaccine and any combination thereof. In some embodiments, the modified T cell and the additional agent are co-administered to the subject. In some embodiments, the additional agent is an anti-programmed cell death 1 (PD-1) antibody.

In some embodiments, the subject is a human.

For any of the previous aspects or embodiments, the MCR binding domain may comprise an HA-tag or optionally the HA-tag is absent. In some embodiments, the MCR binding domain further comprises a linker or optionally the linker is absent. In further embodiments, the linker is positioned between the MCR binding domain and the HA-tag.

For any of the previous aspects or embodiments, the CAR comprises an HA-tag or optionally the HA-tag is absent. In some embodiments, the CAR further comprises a linker or optionally the linker is absent. In further embodiments, the linker is positioned between the MCR binding domain and the HA-tag.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B are a series of schematic representations of the various chimeric antigen receptor constructs described in this invention (FIG. 1A) and the amino acid sequences of the ligands of interest: MSH (SEQ ID NO: 1), MS05 (SEQ ID NO: 2), Agouti (SEQ ID NO: 3) and Agouti F117A (SEQ ID NO: 4), (FIG. 1B).

FIG. 2 is a series of graphs demonstrating the high expression of CARs. Flow cytometry analysis showed a high expression of HA-tagged CAR molecules on the surface of a majority of transduced human T-cells as compared to nontransduced (NTD) control cells.

FIG. 3 is a series of histograms showing CAR-T cells with MSH or Agouti F117A targeting domains selectively kill melanocytes through time at 24 hours. Fibroblasts are unaffected by the CAR-T cells. RLU: Relative Luminescence Units (luciferase activity), MC: melanocytes, FB: fibroblasts, NTD: nontransduced control T cells, NP40: lysis control. MC and FB target cells were donor matched (identical HLA).

FIG. 4 is a series of histograms showing CAR-T cells with MSH or Agouti F177A targeting domains display continued selective killing of melanocytes at 32 hours. Fibroblasts are unaffected by the CAR-T cells. Fibroblasts are unaffected by the CAR-T cells. RLU: Relative Luminescence Units (luciferase activity), MC: melanocytes, FB: fibroblasts, NTD: nontransduced control T cells, NP40: lysis control. MC and FB target cells were donor matched (identical HLA).

FIG. 5 is a histogram showing that AgF117A T-cells kill efficiently melanoma cells. A Lactate de-hydrogenase (LDH) assay was performed: LDH (a protein found on the inside of cells) is released when cells lyse and die. LDH release can be detected by a commercially available kit and the more LDH is released is the more cell are killed. The present assay showed that AgF117A T-cells significantly increase LDH release from human WM46 melanoma cells, while having little toxicity on other cell types.

FIG. 6 is a histogram showing that AgF117A T-cells kill efficiently melanoma cells while having little toxicity on other cell types. A Lactate de-hydrogenase (LDH) assay was performed with the same conditions as FIG. 5 above. The present assay showed that AgF117A T-cells significantly increase LDH release from human WM46 melanoma cells, while having little toxicity on fibroblasts and melanocytes.

FIG. 7 is a series of images of cell cultures (Fibroblasts vs. human melanoma cells (WM46) demonstrating that AgF117A T-cells significantly decrease the number of live human WM46 melanoma cells compared to control non-transduced T cell-treated cultures or live cells alone, while having no detectable toxicity on HLA-matched fibroblasts.

FIG. 8 is a chart illustrating the experimental set up for the AgF117A T-cells in vivo. The timeline used was the following: Day 0: Inject WM46 cells; Day 21: Measure tumors, randomize mice, inject control or AgF117A T cells; Day 28: Measure tumors; Day 35: Measure tumors and Day 39: Measure tumors.

FIG. 9 is a graph showing that AgF117A T-cells cause regression of WM46 tumors in NSG mice. Following CAR treatment, the average tumor growth was measured.

FIG. 10 is a series of photographs depicting how AgF117A T-cells cause regression of WM46 tumors in NSG mice: Tumors at baseline (top photographs), Control T-cell treated tumors at 2 weeks, 4 days treatment (middle photographs) and AgF117A T-cell treated tumors at 2 weeks, 4 days treatment (bottom photographs).

FIG. 11 is a series of micrographs illustrating co-culture of CAR-T cells (control or AgF117A) with WM2664 human melanoma, SKMEL-2 human melanoma, HPAC human pancreatic cancer, and HPAC cells overexpressing MC1R, the antigen of interest. Left column labeled “Cells only” is cancer cells without any CAR-T-cells present.

FIG. 12 is a graph illustrating Xcelligence cell death assay results. MC1R negative MIAPACA2 human pancreatic cancer cells and MC1R positive SKMEL2 human melanoma cells were cocultured with control or AgF117A CAR-T cells (each condition in triplicate). Readings of adherent (target) cell death were conducted every 20 minutes. AgF117A CAR-T were cytotoxic to MC1R+ SKMEL2 cells but not MC1R-MIAPACA cells.

FIG. 13 is a graph illustrating that AgF117A CAR-T cells kill BRAF driven human melanoma. A single infusion of AgF117A T-cells caused an initial regression of WM46 tumors in NSG mice and extended survival. Tumor volumes shown on log scale. Dashed line-all control T cell treated mice euthanized due to tumor volume by 6 weeks after T cell injection, while 3/5 AgF117A CAR-T treated mice survived for 12 weeks. *=Spontaneous deaths.

FIG. 14 is a graph illustrating that AgF117A CAR-T cells kill NRAS driven human melanoma. A single infusion of AgF117A T-cells caused an initial regression of SKMEL2 tumors in NOD-SCID mice. Tumor volumes are shown on log scale, time elapsed since T-cell infusion on the horizontal axis

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “MCR peptide ligand” as used herein refers to a molecule, fragment of a molecule, peptides, or a polypeptide sequence that binds to a MCR. The MCR peptide ligand can be a MCR agonist (e.g. MSH) or a MCR antagonist (e.g. Agouti).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, ovarian cancer, renal cell carcinoma, bladder cancer, kidney cancer, testicular cancer, prostate cancer, breast cancer, colon cancer, pancreatic cancer, lung cancer, liver cancer, stomach, thyroid cancer, and the like.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting the specificity of a T cell expressing the CAR specific for tumor associated antigens.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for the ability to bind MCR using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

The term “dysregulated” when used in the context of the level of expression or activity of MCR refers to the level of expression or activity that is different from the expression level or activity of MCR in an otherwise identical healthy animal, organism, tissue, cell or component thereof. The term “dysregulated” also refers to the altered regulation of the level of expression and activity of MCR compared to the regulation in an otherwise identical healthy animal, organism, tissue, cell or component thereof.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the phrase “MCR binding domain” refers to a protein domain or polypeptide that specifically binds to a melanocortin receptor (MCR). In one embodiment, the MCR binding domain may comprise MCR peptide ligand or fragment thereof, a MCR antagonist or fragment thereof, or an anti-MCR agonist or fragments thereof.

As used herein, “MCR antagonist” refers to a molecule or fragment thereof that has affinity for a melanocortin receptor (MCR). The MCR antagonist has affinity to the active site on MCR, a similar or the same binding site as a melanocyte stimulating hormone (e.g. aMSH). MCR antagonist binding affinity to the MCR may be reversible or irreversible.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The phrases “an immunologically effective amount”, “an anti-immune response effective amount”, “an immune response-inhibiting effective amount”, or “therapeutic amount” refer to the amount of the composition of the present invention to be administered to a subject which amount is determined by a physician, optionally in consultation with a scientist, in consideration of individual differences in age, weight, immune response, type of disease/condition, and the health of the subject (patient) so that the desired result is obtained in the subject.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

“Melanocortin receptor (MCR)” is a G protein-coupled receptor. As used herein MCR refers to the family of human melanocortin receptors comprising melanocortin 1 receptor (MC1R), melanocortin 2 receptor (MC2R), melanocortin 3 receptor (MC3R), melanocortin 4 receptor (MC4R), and melanocortin 5 receptor (MC5R).

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell. An example of a “cell surface receptor” is human MCR.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an WIC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target cell” or “target site” refers to a cell or site to which a binding molecule may specifically bind under conditions sufficient for binding to occur. Binding may occur through a molecule or fragment thereof, such as an antigen, on the target cell or at a target site to a binding partner, such as an antibody.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention includes compositions and methods for using chimeric receptors that specifically bind to melanocortin receptor (MCR). The present invention relates generally to the treatment of a patient having a melanoma. The invention also includes methods of making a genetically modified T cell expressing a chimeric antigen receptors (CAR) that bind to MCR.

### MCR CARs

The present invention includes an isolated chimeric antigen receptor (CAR) comprising a melanocortin receptor (MCR) binding domain that comprises a MCR peptide ligand or fragment thereof, a MCR antagonist or fragment thereof, or an anti-MCR agonist or fragment thereof. The invention also includes methods of making such a CAR. The CAR of the invention can be incorporated into an immunotherapy, a pharmaceutical composition, and the like. Accordingly, the present invention provides compositions and methods for treating melanomas.

In one aspect, the invention includes an isolated nucleic acid sequence encoding a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof. In one embodiment, the CAR is encoded by a nucleic acid sequence selected from the group consisting of SEQ ID NOs: 11, 12, 15, 16, 19, and 20. In another embodiment, the invention includes a vector comprising the isolated nucleic acid sequence encoding the MCR CAR described herein.

In another aspect, the invention includes isolated CAR comprising a melanocortin receptor (MCR) binding domain, a transmembrane domain, a costimulatory signaling region, and a signaling domain, wherein the MCR binding domain comprises a MCR peptide ligand or fragment thereof. In one embodiment, CAR comprises an amino acid sequence selected from the group consisting of SEQ ID NOs: 13, 14, 17, 18, 21 and 22. In another embodiment, a cell comprises the isolated MCR CAR described herein.

In one aspect, the invention includes an isolated nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

In another aspect, the invention includes an isolated chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

In another aspect, the invention includes a modified cell comprising a nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

In yet another aspect, the invention includes a modified cell comprising a nucleic acid sequence encoding a CAR comprising a MCR binding domain, a transmembrane domain, a costimulatory signaling region, and a signaling domain, wherein the MCR binding domain comprises a MCR peptide ligand or fragment thereof. In one embodiment, the cell is selected from a T cell, a natural killer (NK) cell, a cytotoxic T lymphocyte (CTL), a regulatory T cell, and a macrophage.

In one embodiment, the MCR binding domain specifically binds to MCR expressed on melanocytes and melanocytic tumors, or on tumors arising in other organs including, but not limited to, brain.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than as cloned molecules. In one embodiment, the CAR of the invention comprises a target-specific binding element otherwise referred to as an antigen binding moiety as described elsewhere herein. Examples of cell surface markers that may act as ligands for the antigen moiety domain in the CAR of the invention include those associated with viral, bacterial and parasitic infections, autoimmune disease and cancer cells.

MCR Binding Domain

In one embodiment, the MCR binding domain portion of the CAR targets MCR, including human MCR. The choice of MCR binding domain encompasses domains that specifically bind to MCR. For example, the MCR binding domain may include a MCR peptide ligand or fragment thereof, a MCR antagonist or fragment thereof, or an anti-MCR agonist or fragment thereof. In some embodiments, the MCR comprises any MCR known in the art such as, but not limited to, melanocortin 1 receptor (MC1R), melanocortin 2 receptor (MC2R), melanocortin 3 receptor (MC3R), melanocortin 4 receptor (MC4R), and melanocortin 5 receptor (MC5R). In other embodiments, the MCR is MCR1.

In one embodiment, the MCR binding domain comprises an amino acid sequence derived from a melanocortin (MC) molecule. The MCR binding domain includes fragments, peptides, or polypeptide sequences derived from a MC hormone molecule. In one embodiment, the MCR is MC1R. In another embodiment, the MCR binding domain comprises at least one MCR peptide ligand selected from the group consisting of: an alpha-melanocyte stimulating hormone (aMSH), an Agouti protein (Ag), an Agouti-related protein (AgRP) and any mutant or variant thereof such as, but not limited to, aMSH mutant (MS05) and Agouti F117A mutant which are both highly specific for MC1R. In some embodiments, the MCR binding domain is encoded by a nucleic acid sequence selected from the group consisting of SEQ ID NOs: 5, 7 and 9. In other embodiments, the MCR binding domain comprises an amino acid sequence selected from the group consisting of SEQ ID NOs: 1-4, 6, 8 and 10.

The MCR binding domain may include any fragment of a melanocyte stimulating hormone (MSH) or of an Agouti molecule. In some embodiments, the MCR binding domain comprises at least 10 amino acids in length of a MSH or an Agouti molecule. The MCR binding domain may include at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids of a MSH or an Agouti molecule. In one embodiment, the MCR binding domain comprises about 6 to about 40 amino acids of a MSH or an Agouti molecule. In another embodiment, the MCR binding domain comprises about 10 to about 30 amino acids of a MSH or an Agouti molecule. In yet another embodiment, the MCR binding domain comprises about 15 to about 25 amino acids of a MSH or an Agouti molecule. In still another embodiment, the MSH or Agouti fragment retains the capacity to bind to a MCR.

The MCR binding domains includes MCR peptide ligands with homologous domains that may have 80%, 81%, 82%, 83%, 84%, 85%, 86%, 87%, 88%, 89%, 90%, 91%, 92%, 93%, 94%, 95%, 96%, 97%, 98%, 99%, or greater homology to the MCR peptides described herein.

The MCR binding domain is encoded by a nucleic acid encoding a MCR binding domain derived from a MSH molecule. The nucleic acid encoding a MCR binding domain includes nucleotide sequences or fragments thereof derived from a nucleic acid encoding a MSH molecule. In one embodiment, the nucleic acid encoding a MCR binding domain comprises a nucleotide sequence or fragment thereof encodes an anti-MCR peptides. The MCR may be encoded by a nucleic acid encoding a MCR binding domain comprising a nucleic acid sequence having 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleic acid encoding the anti-MCR peptides described herein.

The MCR binding domain may include a MCR antagonist or fragment thereof, or an anti-MCR agonist or fragment thereof. Example of MCR antagonists and anti-MCR agonists include, but are not limited to, αMSH, Agouti (Ag), Agouti-related proteins (AgRPs) and β-defensin 3 (BD3).

In another embodiment, the MCR binding domain specifically binds to MCR expressed on tumors cells.

Transmembrane Domain

The CAR comprises a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Signaling Domain and Costimulatory Domain

The signaling domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs.

Examples of ITAM containing primary signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that signaling molecule in the CAR of the invention comprises a signaling domain derived from CD3-zeta.

In one embodiment, the signaling domain of the CAR can be designed to comprise the CD3-zeta signaling domain by itself or combined with any other desired cytoplasmic domain(s) useful in the context of the CAR of the invention. For example, the signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, CD5, ICAM-1, GITR, BAFFR, HVEM (LIGHTR), SLAMF7, NKp80 (KLRF1), CD127, CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof. Thus, while the invention in exemplified primarily with CD8 and 4-1BB (CD137) as the co-stimulatory signaling domains, other costimulatory domains are within the scope of the invention.

The signaling domains within the intracellular portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the signaling domain is designed to comprise the signaling domain of CD3-zeta and the signaling domain of CD8. In another embodiment, the signaling domain is designed to comprise the signaling domain of CD3-zeta and the signaling domain of CD137 (4-1BB).

### MCR CAR

The present invention therefore encompasses a nucleic acid sequence encoding a CAR comprising a melanocortin receptor (MCR) binding domain, a transmembrane domain and an intracellular signaling domain. The nucleic acid sequence may include a recombinant DNA construct comprising sequences of an antibody that specifically binds to MCR, wherein the sequence of the antibody or a fragment thereof is operably linked to the nucleic acid sequence of an intracellular domain. The intracellular domain or otherwise the cytoplasmic domain comprises, a costimulatory signaling region and/or a zeta chain portion. The costimulatory signaling region refers to a portion of the CAR comprising the intracellular domain of a costimulatory molecule.

In one aspect, the invention includes an isolated nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In one embodiment, a cell comprises the isolated nucleic acid sequence encoding the CAR described herein.

In another aspect, the invention includes an isolated chimeric antigen receptor (CAR) comprising a melanocortin receptor (MCR) binding domain, a transmembrane domain, a costimulatory signaling region, and a CD3 zeta signaling domain, wherein the MCR binding domain comprises anti-MCR antibody or a fragment thereof. In one embodiment, a cell comprises the isolated CAR described herein.

In still yet another aspect, the invention includes a modified cell comprising a nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof. In one embodiment, the cell is selected from a T cell, a natural killer (NK) cell, a cytotoxic T lymphocyte (CTL), and a regulatory T cell.

In one embodiment, the CAR specifically binds to MCR expressed by tumor cells and/or tumor vasculature. In another embodiment, the MCR binding domain of the CAR specifically binds to MCR expressed on tumor cells. The tumor cells may include cells from a melanoma or cells from a cancer such as but not limited to skin cancer.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

The nucleic acid sequences encoding the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than as cloned molecules. In one embodiment, the CAR of the invention comprises a target-specific binding element otherwise referred to as an antigen binding moiety as described elsewhere herein. Examples of cell surface markers that may act as ligands for the antigen moiety domain in the CAR of the invention include those associated with viral, bacterial and parasitic infections, autoimmune disease and cancer cells.

MCR Binding Domain

In a preferred embodiment, the MCR binding domain portion of the CAR targets MCR, including human MCR. The choice of MCR binding domain encompasses domains that specifically bind to MCR. For example, the MCR binding domain may include antibodies that specifically bind MCR. MCR antibodies are described in more detail elsewhere herein.

The MCR binding domain can be any domain of an antibody that binds to MCR including, but not limited to, monoclonal antibodies, polyclonal antibodies, synthetic antibodies, human antibodies, humanized antibodies, single fragment variable chains (scFv), and fragments thereof. Thus, in one embodiment, the MCR binding domain of the CAR comprises a human antibody or a fragment thereof. In another embodiment, the MCR binding domain is an antibody selected from the group consisting of a human antibody, humanized antibody, and fragment thereof. In yet another embodiment, the MCR binding domain comprises a heavy and light chain. In still another embodiment, the MCR binding domain is selected from the group consisting of a Fab fragment, a F(ab′)₂ fragment, a Fv fragment, and a single chain Fv (scFv).

### Anti-MCR Antibodies

The antibodies of the invention are characterized by particular functional features or properties. For example, the antibodies specifically bind to human MCR. In some embodiments, the antibodies bind to MCR with high affinity. The antibodies of the invention may specifically recognize naturally expressed MCR protein on a cell. It may also be advantageous if the anti-MCR antibodies do not cross-react with other surface molecules.

In one embodiment, the antibody contains heavy chain variable regions having CDRs 1, 2 and 3. In one embodiment, the antibody contains light chain variable regions having CDRs 1, 2 and 3.

Given that each of these antibodies binds to MCR, the V_{H} and V_{L} sequences can be “mixed and matched” to create other anti-MCR binding molecules of the invention. MCR binding of such “mixed and matched” antibodies can be tested using the binding assays described herein, in the art, for example, in the Examples section (e.g., ELISAs). Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence. It will be readily apparent to the ordinarily skilled artisan that novel VH and VL sequences can be created by substituting one or more VH and/or VL CDR region sequences with structurally similar sequences from the CDR sequences disclosed herein.

In one embodiment, the invention includes antibodies that comprise the heavy chain and light chain CDR1s, CDR2s and CDR3s. In certain embodiments, the antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences or conservative modifications thereof, and wherein the antibodies retain the desired functional properties of the anti-MCR antibodies of the invention. Accordingly, the invention provides an isolated antibody (e.g., scFv), or antigen binding portion thereof, comprising a heavy chain variable region comprising CDR1, CDR2, and CDR3 sequences and a light chain variable region comprising CDR1, CDR2, and CDR3 sequences.

In another embodiment, the invention includes antibodies that bind to the same epitope on human MCR as any of the MCR antibodies of the invention (i.e., antibodies that have the ability to cross-compete for binding to MCR with any of the antibodies of the invention

An antibody of the invention is prepared using an antibody having one or more of the VH and/or VL sequences disclosed herein as a starting material to engineer a modified antibody, which modified antibody may have altered properties as compared with the starting antibody. An antibody can be engineered by modifying one or more amino acids within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

In certain embodiments, the invention comprise a small peptide that comprises specific amino acids of a MSH or an Agouti molecule and target MCR. In certain embodiments, the peptide comprises 6-40 amino acids. In other embodiments, peptide comprises 10-30 amino acids. In other embodiments, peptide comprises 15-25 amino acids. The invention also provides methods of making such immuno-receptors.

### Human Antibodies

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology

(McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Humanized Antibodies

Alternatively, in some embodiments, a non-human antibody is humanized, where specific sequences or regions of the antibody are modified to increase similarity to an antibody naturally produced in a human. In one embodiment, the antigen binding domain is humanized.

A “humanized” antibody retains a similar antigenic specificity as the original antibody. However, using certain methods of humanization, the affinity and/or specificity of binding of the antibody for human CD3 antigen may be increased using methods of “directed evolution,” as described by Wu et al., J. Mol. Biol., 294:151 (1999), the contents of which are incorporated herein by reference herein in their entirety.

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

### Vectors

The present invention also provides vectors in which the isolated nucleic acid sequence of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., Molecular Cloning: A Laboratory Manual, volumes 1-3 (3^{rd} ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the EF1alpha promoter. An additional example includes the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, volumes 1-3 (3^{rd} ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Controlled Expression of MCR CAR

The present invention also contemplates the use of CAR technology for methods of selectively ablating or activating modified T cells from a subject after adoptive transfer. In one embodiment, a modified CAR T cell is selectively ablated in the subject by inducing activation of a suicide gene in the modified T cell. In another embodiment, a modified T cell is selectively activated in the subject by actively preventing the apoptosis death or dysfunction of modified CAR T cell during therapy. In yet another embodiment, the modified T cell is selectively activated in the subject by allowing cell-surface expression of the CAR construct.

### Suicide Gene

Some of the potential side effects of non-target cell recognition by CAR T cells can be overcome by the co-expression of a suicide gene in the CART cell.

Thus, in some embodiments, the MCR CAR of the invention includes an isolated nucleic acid comprising a suicide gene. Examples of suicide genes include, but are not limited to, herpes simplex virus thymidine kinase (HSV-TK), the cytoplasmic domain of Fas, a caspase such as caspase-8 or caspase-9, cytosine deaminase, E1A, FHIT, and other known suicide or apoptosis-inducing genes (Straathof et al., 2005, Blood 105:4247-4254; Cohen et al., 1999, Leuk. Lymphoma 34:473-480; Thomis et al., 2001, Blood 97:1249-1257; Tey et al., 2007, Biol. Blood Marrow Transplant 13:913-924; and Di Stasi et al., 2011, N. Engl. J. Med. 365:1673-1683).

The suicide gene may be operably linked to a promoter, such as an inducible promoter sequence. Examples of inducible promoters include, but are not limited to, a heat shock promoter, a tetracycline-regulated promoter, a steroid-regulated promoter, a metal-regulated promoter, an estrogen receptor-regulated promoter, and others known in the art. In one aspect, the invention includes an isolated nucleic acid sequence comprising a nucleic acid sequence comprising a suicide gene and a nucleic acid encoding a chimeric antigen receptor. In another aspect, the invention includes an isolated nucleic acid sequence comprising a nucleic acid sequence comprising a suicide gene and a nucleic acid encoding a chimeric antigen receptor.

In yet another embodiment, the suicide gene has an inducible promoter.

In some embodiments, the suicide gene is in an expression vector. The expression vector may also include other genes, such as a MCR CAR and/or a CRISPR system as described elsewhere herein.

The invention also includes a cell comprising the suicide gene. In an exemplary aspect, the present invention includes a modified cell comprising a nucleic acid comprising a suicide gene and a nucleic acid encoding a CAR In another aspect, the present invention includes a modified cell comprising a nucleic acid comprising a suicide gene and a nucleic acid encoding a CAR.

In one embodiment, the MCR CAR modified T cell comprises nucleic acids encoding a suicide gene as a separate nucleic acid sequence from the CAR construct described elsewhere herein. For example, HSV-TK, i-Casp9, the cytoplasmic domain of Fas, or a caspase can be incorporated into genetically engineered T cells separate from the CAR construct. In another embodiment, the MCR CAR modified T cell comprises a suicide gene in the same construct as the nucleic acids encoding the CAR. In this embodiment, the nucleic acids comprising the suicide gene may be upstream or downstream of the nucleic acids encoding the CAR.

In one embodiment, expression of the suicide gene is activated in the cell by contacting the cell with an inducing agent administered to the cell or to a mammal comprising the cell. The inducing agent then activates an inducible promoter to express the suicide gene. In such an embodiment, the inducing agent is administered to the subject to induce expression of the suicide gene.

In another embodiment, a suicide gene product that is expressed from the suicide gene is activated by an activating agent, such as a dimerization agent. For example, the dimerization agent, such as AP20187, promotes dimerization and activation of caspase-9 molecules.

In some embodiments with constitutive expression of the suicide gene, expression of the suicide gene may be turned off in the cell by contacting the cell with an inhibiting agent administered to the cell or to a mammal comprising the cell. The inhibiting agent selectively turns off expression. For example, caspase-9 is constitutively expressed in the cell and the addition of an inhibiting agent represses expression or activation of caspase-9. In one embodiment, the inhibiting agent is administered to the subject to repress expression of the suicide gene.

In some embodiments with constitutive expression of the suicide gene, activation of the suicide gene product may be repressed in the cell by contacting the cell with an inhibiting agent, such as a solubilizing agent, administered to the cell or to a mammal (e.g. a human) comprising the cell. The inhibiting agent represses activation of the suicide gene product, such as by preventing dimerization of the caspase-9 molecules. In one embodiment, the solubilizing agent is administered to the subject to repress activation of the suicide gene product.

In some aspects, the suicide gene is not immunogenic to the cell comprising the suicide gene or host harboring the suicide gene. Although thymidine kinase (TK) may be employed, it can be immunogenic. Alternatively, examples of suicide genes that are not immunogenic to the host include caspase-9, caspase-8, and cytosine deaminase.

In yet another embodiment, suicide gene expression is linked in tandem to dimerization domains, which cause aggregation and degradation of the transcript, preventing cell-surface expression and hence function of the suicide gene. Solubilization of the dimerization domains with a solubilizing agent, administered to the cell or to a mammal comprising the cell, prevents aggregation and allows the construct to egress through the secretory system.

In some embodiments, the MCR CAR further comprises a dimerization domain, such a dimerization domain from FKBP or similar molecule. In such an embodiment, the presence of CAR molecules on the surface of the modified T cell is prevented by spontaneous aggregation of the CAR molecules in the cytoplasm or other internal location in the cell. Solubilization of the dimerization domains with a solubilizing agent, administered to the cell or to a mammal comprising the cell, prevents dimerization and allows the construct to egress through the secretory system, where it is processed via furin cleavage to express a functional cell surface CAR.

In the presence of a solubilizing agent, the CAR molecules are separated and aggregation is prevented. Administration of a solubilizing agent would prevent dimerization or aggregation of the CAR molecules and allow the CAR molecules to be presented on the surface of the T cell.

### CRISPR/Cas

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/CAS system can thereby be engineered to cleave virtually any DNA sequence by redesigning the gRNA in cell lines (such as 293T cells), primary cells, and CAR T cells. The CRISPR/CAS system can simultaneously target multiple genomic loci by co-expressing a single CAS9 protein with two or more gRNAs, making this system uniquely suited for multiple gene editing or synergistic activation of target genes.

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, the CRISPR system comprises an expression vector, such as, but not limited to, an pAd5F35-CRISPR vector. In one embodiment, the modified T cell described herein is further modified by introducing a Cas expression vector and a guide nucleic acid sequence specific for a gene into the modified T cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. The guide nucleic acid sequence may be specific for a T cell receptor (TCR) chain (such as an alpha, beta, gamma and/or delta chain), a major histocompatibility complex protein (such as a HLA class I molecule and/or HLA class II molecule), and any combination thereof. The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In some embodiments, a T cell is modified to express a CAR and the CAR T cell is further modified to delete endogenous TCR or MHC molecules, such as before administration to a subject. In one embodiment, the CAR modified T cell described herein is further modified by deleting a gene selected from the group consisting of a T cell receptor (TCR) chain, a major histocompatibility complex protein, and any combination thereof. In another embodiment, the T cell is modified before administration to the subject in need thereof.

In some embodiments, a T cell is modified to express a CAR, administered to a subject, and then further modified in vivo to delete endogenous TCR or MHC molecules, such as through inducing targeted gene deletion. In one embodiment, the modified T cell described herein is modified by inducing a CRISPR/Cas system to minimize native reactivity of the modified T cell or host reactivity to the modified T cell. In some embodiments, inducing the Cas expression vector comprises exposing the modified T cell to an agent that activates an inducible promoter in the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In some embodiments, a T cell is modified to delete endogenous TCR or MHC molecules prior to modification to express the CAR. In some embodiments, the modified T cell is further modified by deleting TCR or MHC molecules prior to inducing expression of the suicide gene.

### Vectors

The present invention also provides vectors in which the isolated nucleic acid sequence of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., Molecular Cloning: A Laboratory Manual, volumes 1-3 (3^{rd} ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the EF1alpha (EF1α) promoter. An additional example includes the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, volumes 1-3 (3^{rd} ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL gradient or by counterflow centrifugal elutriation. A specific subpopulation of T cells, including, but not limited to CD3⁺, CD28⁺, CD4⁺, CD8⁺, CD45RA⁺, CD45RO⁺T, CD26L+, CCR7+ cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar method of selection. In other embodiments, subpopulation of T cells, such as, but not limited to, cells positive or expressing high levels of one or more surface markers e.g. CD28+, CD8+, CCR7+, CD27+, CD127+, CD45RA+, and/or CD45RO+ T cells, can be isolated by positive or negative selection techniques.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells are activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

The isolated nucleic acid sequences described herein, the encoded MCR CAR, or cells comprising either the isolated nucleic acid sequences or the encoded MCR CAR are included in a composition for therapy. The composition may include a pharmaceutical composition and further include a pharmaceutically acceptable carrier. A therapeutically effective amount of the pharmaceutical composition comprising the MCR CAR is administered to a patient in need thereof.

In one aspect, the invention includes a method for stimulating a T cell-mediated immune response to a melanocyte-derived cell population in a mammal. The method comprises administering to a subject an effective amount of a modified cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof.

In one aspect, the invention includes a method for stimulating a T cell-mediated immune response to a melanocyte-derived cell population in a mammal. The method comprises administering to a subject an effective amount of a modified cell that expresses a chimeric antigen receptor (CAR) comprising a melanocortin receptor (MCR) binding domain, a transmembrane domain, a costimulatory signaling region, and a signaling domain, wherein the MCR binding domain comprises an anti-MCR antibody or a fragment thereof.

In another aspect, the invention includes a method of treating a subject with a cancer. The method comprises administering to the subject a modified T cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof.

In yet another aspect, the invention includes a method of treating a subject with a cancer. The method comprises administering to the subject a modified T cell that expresses a chimeric antigen receptor (CAR) comprising a melanocortin receptor (MCR) binding domain, a transmembrane domain, a costimulatory signaling region, and a signaling domain, wherein the MCR binding domain comprises an anti-MCR antibody or a fragment thereof. In one embodiment, the cancer is a melanoma arising from skin or other tissues. In another embodiment, the modified T cell is autologous to the subject. In another embodiment, the method further includes administering to the subject an additional agent selected from the group consisting of a chemotherapeutic agent, an anti-cell proliferation agent, an immunotherapeutic agent, an antitumor vaccine and any combination thereof. In some embodiments, the modified T cell and the additional agent are co-administered to the subject. In some embodiments, the additional agent is an anti-programmed cell death 1 (PD-1) antibody.

In another embodiment, the cells described herein may be used for the manufacture of a medicament for the treatment of an immune response in a subject in need thereof. In yet another embodiment, the cells described herein may be used for the manufacture of a medicament for the treatment of a cancer or a disease, disorder and condition associated with dysregulated expression of MCR in a subject in need thereof. In one embodiment the subject is a mammal. In another embodiment the subject is a human.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

The present invention includes a type of cellular therapy where cells are genetically modified and infused to a recipient in need thereof. In one embodiment, a method is disclosed for adoptive transfer therapy comprising administering a population of the modified cells to a subject in need thereof. The cells are able to kill the diseased cells (e.g. melanocytic tumor cells) in the subject. Unlike traditional antibody therapies, the modified cells described herein are able to replicate in vivo resulting in long-term persistence that can lead to sustained disease control. In various embodiments, the cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cells to the patient.

The fully-human or humanized CAR-modified cells of the invention may be a type of vaccine for ex vivo immunization and/or in vivo therapy in a mammal. Particularly, the compositions and methods of the invention may be used for in vivo immunization to elicit an immune response directed against an MCR antigen in a mammal. Preferably, the mammal is a human.

Ex vivo procedures are well known in the art as discussed more fully above. Briefly, cells are isolated from a mammal (preferably a human) and genetically modified (i.e., transduced or transfected in vitro) with a vector comprising the isolated nucleic acid sequence disclosed elsewhere herein. The modified cells can be administered to a mammalian recipient to provide a therapeutic benefit. The mammalian recipient may be a human and the modified cells can be autologous with respect to the recipient. Alternatively, the cells can be allogeneic, syngeneic or xenogeneic with respect to the recipient.

Generally, the cells may be activated and expanded as described herein then utilized in the treatment and prevention of diseases that arise in individuals who are immunocompromised. In particular, the modified cells of the invention are used in the treatment of a cancer or a disease, disorder and condition associated with dysregulated expression of MCR. In certain embodiments, the cells of the invention are used in the treatment of patients at risk for developing a cancer or a disease, disorder and condition associated with dysregulated expression of MCR. Thus, the present invention provides methods for the treatment or prevention of diseases, disorders and conditions associated with dysregulated expression of MCR.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogeneic with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i. v.) injection, or intraperitoneally. In other instances, the cells of the invention are injected directly into a site of inflammation in the subject, a local disease site in the subject, a lymph node, an organ, a tumor, and the like.

The cells described herein can also be administered using any number of matrices. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized, as such these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

### Pharmaceutical Compositions

Pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 ml to 400 ml. In certain embodiments, T cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of T cells.

The administration of the subject compositions may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Design and Construction of the MSH, MS05, Agouti and Agouti F117A CARs

CARs were cloned by inserting codon-optimized synthesized DNA (GeneArt, Life Technologies, or IDT) into a third generation lentiviral vector, pRRLSIN.cPPT.PGK-GFP.WPRE (Addgene #12252), in which the PGK promoter was first replaced with the human EF1a promoter. The vector backbone was modified to also contain the CD8a signal peptide, hinge and transmembrane as well as the CD137 and CD3zeta domains, so that cloning of the MC1R ligands was performed using BamHI (3′ of the CD8a signal peptide) and AccIII (isoschizomer: BspEI; 5′ of the CD8 hinge). DNA fragments and vector backbone were digested at 37° C. for 1 hour, followed by PCR- or gel-purification, respectively. Inserts were ligated into the vector for 1 hour at room temperature and the ligated plasmids were transformed into Top10 chemically competent cells (ThermoFisher). All constructs were sequence-verified before proceeding with downstream applications.

### Lentiviral Production

VSV-G pseudotyped lentiviral particles were produced using a 4th generation packaging system. 293T cells (as these are commonly used for lentivirus production) were transfected at a confluency of 90% with a mixture of the pRRLSIN.cPPT.EF1α-gene-of-interest. WPRE, the envelope plasmid pMD2.G (Addgene #12252), the packaging plasmids pRSVRev (Addgene #12253) and pMDLgm/pRRE (Addgene #12251) were assembled in a complex with Lipofectamine 2000 (Life Technologies). Lentivirus containing supernatant was harvested after 24 and 48 hours, filtered through a 0.4 micron PES membrane, concentrated at 12,000×g for 12 hours at 4° C. and stored at −80° C.

### Stimulation and Expansion of Primary Human T Cells

Primary human T cells were cultured in RPMI1640, 10% FBS, 10 mM HEPES, 1% penicillin/streptomycin. Bulk T cells (CD4⁺ and CD8⁺) were stimulated with anti-CD3 and anti-CD28 beads (Dynabeads, Life Technologies) at a bead:cell ratio of 3:1. The culture medium was supplemented with 100 IU/mL interleukin 2. 24 hours after stimulation, 10⁶ T cells were transduced with CAR or control constructs or mock transduced. Expansion of the T cells was monitored for 8-12 days by measurement of cell volume and concentration (Coulter counter, Beckman Coulter). Cell surface expression of the CAR constructs was validated by flow cytometry (BD LSRII) with a monoclonal antibody against the HA-tag (clone 3F10, Roche). CAR expression was quantified by staining with aforementioned antibody for 25 minutes at room temperature. Subsequently, after washing twice, bound antibodies were detected with donkey anti-rat IgG AF594 (ThermoFisher). All staining procedures were performed under saturating conditions. For some experiments, transduced T cells were previously frozen in 90% FBS and 10% DMSO. Experiments were performed when cells were rested after stimulation (i.e. volume of <4504

### Luciferase Assay

Primary human melanocytes and fibroblasts transduced with firefly luciferase were plated in triplicate at 50% confluence on 96 well plates. The next day, CAR-T cells were added in a 5:1 T-Cell:target cell ratio. Media was 50% RPMI with 50% melanocyte media (for MC) or 50% DMEM with 5% FBS (for fibroblasts). After 24 or 36 hours D-luciferin (150 ug/ml final concentration) in PBS was added and luciferase activity determined on a standard luminescence plate reader.

### LDH Assay

WM46 human melanoma cells, primary human fibroblasts, and primary human melanocytes were plated in triplicate at 50% confluence on 96 well plates. The next day, CAR-T cells were added in a 5:1 T-Cell:target cell ratio. Media was 50% RPMI with 50% melanocyte media (for MC), 50% RPMI with 50% Tu2% (for WM46) or 50% RPMI with 50% DMEM (for fibroblasts). After 24 hours of co-culture, LDH release was detected by a commercially available kit (Pierce) and cytotoxicity calculated according to manufacturer instructions.

### Xcelligence Assay

Human MIAPACA2 and SKMEL2 cells were plated at a density of 10,000 cells per well in a 96 well “E-plate” (a 96 well tissue culture plate with a conductive grid on the bottom so it may be analyzed by the Xcelligence apparatus). The next day, CAR-T cells were added in a 5:1 T-Cell:target cell ratio. Automated readings taken by Xcelligence every 20 minutes for 50 hours, and % cytotoxicity analyzed using Xcelligence companion software.

### Nucleotide and Corresponding Amino Acid Sequences of MSH and Agouti Based Targeting Domains:

### Ligands:

1) Agouti sequence (c-terminal of signal peptide
to HA-tag*, nucleotides)
(SEQ ID NO: 5):
CACCTGCCACCAGAAGAAAAGCTCAGAGACGATCGCTCCCTTCGAAGCAA
CAGTAGTGTGAATCTCCTGGACGTGCCCTCCGTGTCTATCGTCGCCCTCA
ATAAAAAGTCCAAACAGATCGGCAGAAAGGCTGCAGAAAAGAAGAGGTCA
AGCAAGAAGGAGGCATCCATGAAAAAGGTCGTCAGGCCTAGGACGCCACT
CTCAGCCCCCTGCGTGGCGACTCGCAACAGCTGCAAACCACCTGCTCCCG
CTTGCTGCGATCCGTGTGCCTCCTGTCAGTGCCGATTTTTTAGATCAGCA
TGTTCATGTCGGGTGCTGTCCCTCAACTGTGGGGGTGGGGGGAGTGGAGG
AGGTGGTGGTGCATACCCGTACGACGTTCCCGATTAC

*HA-tag region (nucleotides or amino acids) as used above and elsewhere herein is underlined and is optional.

Agouti sequence (c-terminal of signal peptide
to HA-tag*, amino acids)
(SEQ ID NO: 6):
HLPPEEKLRDDRSLRSNSSVNLLDVPSVSIVALNKKSKQIGRKAAEKKRS
SKKEASMKKVVRPRTPLSAPCVATRNSCKPPAPACCDPCASCQCRFFRSA
CSCRVLSLNCGGGGSGGGGGAYPYDVPDYA
2) MS05 sequence (c-terminal of signal peptide
to HA-tag*, nucleotides)
(SEQ ID NO: 7):
AGTAGTATTATTAGTCATTTTCGCTGGGGAAAACCTGTTGGAGGAGGGGG
GTCAGGGGGGGGTGGTGGCGCATACCCGTACGACGTTCCCGATTACGCT
MS05 sequence (c-terminal of signal peptide to
HA-tag*, amino acids)
(SEQ ID NO: 8):
SSIISHFRWGKPVGGGGSGGGGGAYPYDVPDYA
3) MSH sequence (c-terminal of signal peptide
to HA-tag*, nucleotides)
(SEQ ID NO: 9):
TCCTACTCCATGGAGCACTTTAGGTGGGGAAAACCCGTCGGGGGTGGGGG
GAGTGGAGGAGGTGGTGGCGCATACCCGTACGACGTTCCCGATTAC
MSH sequence (c-terminal of signal peptide to
HA-tag*, amino acids)
(SEQ ID NO: 10):
SYSMEHFRWGKPVGGGGSGGGGGAYPYDVPDYA
Signal peptide - ligand (Agouti; bold) -CD8
transmembrane CD137-CD247
(SEQ ID NO: 11):
ATGGCCTTACCAGTGACCGCCTTGCTCCTGCCGCTGGCCTTGCTGCTCCA
CGCCGCCAGGCCGGGATCC-CACCTGCCACCAGAAGAAAAGCTCAGAGAC
GATCGCTCCCTTCGAAGCAACAGTAGTGTGAATCTCCTGGACGTGCCCTC
CGTGTCTATCGTCGCCCTCAATAAAAAGTCCAAACAGATCGGCAGAAAGG
CTGCAGAAAAGAAGAGGTCAAGCAAGAAGGAGGCATCCATGAAAAAGGTC
GTCAGGCCTAGGACGCCACTCTCAGCCCCCTGCGTGGCGACTCGCAACAG
CTGCAAACCACCTGCTCCCGCTTGCTGCGATCCGTGTGCCTCCTGTCAGT
GCCGATTTTTTAGATCAGCATGTTCATGTCGGGTGCTGTCCCTCAACTGT
GGGGGTGGGGGGAGTGGAGGAGGTGGTGGTGCATACCCGTACGACGTTCC
CGATTAC-TCCGGAATCTACATCTGGGCCCCTCTGGCCGGCACCTGTGGC
GTGCTGCTGCTGTCCCTGGTCATCACCCTGTACTGCAAGCGGGGCAGAAA
GAAGCTGCTGTACATCTTCAAGCAGCCCTTCATGCGGCCTGTGCAGACCA
CACAGGAAGAGGACGGCTGTAGCTGTAGATTCCCCGAGGAAGAGGAAGGC
GGCTGCGAGCTGAGAGTGAAGTTCAGCAGAAGCGCCGACGCCCCTGCCTA
TCAGCAGGGCCAGAACCAGCTGTACAACGAGCTGAACCTGGGCAGACGGG
AGGAATACGACGTGCTGGACAAGAGAAGAGGCCGGGACCCTGAGATGGGC
GGCAAGCCCAGACGGAAGAACCCCCAGGAAGGCCTGTATAACGAACTGCA
GAAAGACAAGATGGCCGAGGCCTACAGCGAGATCGGCATGAAGGGCGAGC
GGAGAAGAGGCAAGGGCCATGACGGCCTGTACCAGGGCCTGAGCACCGCC
ACCAAGGACACCTACGACGCCCTGCACATGCAGGCCCTGCCTCCAAGATG
A
Signal peptide - ligand (Agouti; bold) -CD8
hinge (italics)-CD8 transmembrane CD137-CD247
(SEQ ID NO: 12):
ATGGCCTTACCAGTGACCGCCTTGCTCCTGCCGCTGGCCTTGCTGCTCCA
CGCCGCCAGGCCGGGATCC-CACCTGCCACCAGAAGAAAAGCTCAGAGAC
GATCGCTCCCTTCGAAGCAACAGTAGTGTGAATCTCCTGGACGTGCCCTC
CGTGTCTATCGTCGCCCTCAATAAAAAGTCCAAACAGATCGGCAGAAAGG
CTGCAGAAAAGAAGAGGTCAAGCAAGAAGGAGGCATCCATGAAAAAGGTC
GTCAGGCCTAGGACGCCACTCTCAGCCCCCTGCGTGGCGACTCGCAACAG
CTGCAAACCACCTGCTCCCGCTTGCTGCGATCCGTGTGCCTCCTGTCAGT
GCCGATTTTTTAGATCAGCATGTTCATGTCGGGTGCTGTCCCTCAACTGT
GGGGGTGGGGGGAGTGGAGGAGGTGGTGGTGCATACCCGTACGACGTTCC
CGATTAC-TCCGGAACCACGACGCCAGCGCCGCGACCACCAACACCGGCG
CCCACCATCGCGTCGCAGCCCCTGTCCCTGCGCCCAGAGGCGTGCCGGCC
AGCGGCGGGGGGCGCAGTGCACACGAGGGGGCTGGACTTCGCCTGTGATT
CCGGAATCTACATCTGGGCCCCTCTGGCCGGCACCTGTGGCGTGCTGCTG
CTGTCCCTGGTCATCACCCTGTACTGCAAGCGGGGCAGAAAGAAGCTGCT
GTACATCTTCAAGCAGCCCTTCATGCGGCCTGTGCAGACCACACAGGAAG
AGGACGGCTGTAGCTGTAGATTCCCCGAGGAAGAGGAAGGCGGCTGCGAG
CTGAGAGTGAAGTTCAGCAGAAGCGCCGACGCCCCTGCCTATCAGCAGGG
CCAGAACCAGCTGTACAACGAGCTGAACCTGGGCAGACGGGAGGAATACG
ACGTGCTGGACAAGAGAAGAGGCCGGGACCCTGAGATGGGCGGCAAGCCC
AGACGGAAGAACCCCCAGGAAGGCCTGTATAACGAACTGCAGAAAGACAA
GATGGCCGAGGCCTACAGCGAGATCGGCATGAAGGGCGAGCGGAGAAGAG
GCAAGGGCCATGACGGCCTGTACCAGGGCCTGAGCACCGCCACCAAGGAC
ACCTACGACGCCCTGCACATGCAGGCCCTGCCTCCAAGATGA
Signal peptide - ligand (Agouti; bold) -CD8
transmembrane CD137-CD247 (amino acids)
(SEQ ID NO: 13):
MALPVTALLLPLALLLHAARPGS-HLPPEEKLRDDRSLRSNSSVNLLDVP
SVSIVALNKKSKQIGRKAAEKKRSSKKEASMKKVVRPRTPLSAPCVATRN
SCKPPAPACCDPCASCQCRFFRSACSCRVLSLNCGGGGSGGGGGAYPYDV
PDYA-SGIYIWAPLAGTCGVLLLSLVITLYCKRGRKKLLYIFKQPFMRPV
QTTQEEDGCSCRFPEEEEGGCELRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPRZ
Signal peptide - ligand (Agouti; bold) -CD8
hinge (italics)-CD8 transmembrane CD137-CD247
(amino acids)
(SEQ ID NO: 14):
MALPVTALLLPLALLLHAARPGS-HLPPEEKLRDDRSLRSNSSVNLLDVP
SVSIVALNKKSKQIGRKAAEKKRSSKKEASMKKVVRPRTPLSAPCVATRN
SCKPPAPACCDPCASCQCRFFRSACSCRVLSLNCGGGGSGGGGGAYPYDV
PDYA-ASTTTPAPRPPTPAPTIASQPLSLRPEACRPAAGGAVHTRGLDFA
CDSGIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRZ
Signal peptide - ligand (M505; bold) -CD8
transmembrane CD137-CD247
(SEQ ID NO: 15):
ATGGCCTTACCAGTGACCGCCTTGCTCCTGCCGCTGGCCTTGCTGCTCCA
CGCCGCCAGGCCGGGATCC-AGTAGTATTATTAGTCATTTTCGCTGGGGA
AAACCTGTTGGAGGAGGGGGGTCAGGGGGGGGTGGTGGCGCATACCCGTA
CGACGTTCCCGATTACGCT-TCCGGAATCTACATCTGGGCCCCTCTGGCC
GGCACCTGTGGCGTGCTGCTGCTGTCCCTGGTCATCACCCTGTACTGCAA
GCGGGGCAGAAAGAAGCTGCTGTACATCTTCAAGCAGCCCTTCATGCGGC
CTGTGCAGACCACACAGGAAGAGGACGGCTGTAGCTGTAGATTCCCCGAG
GAAGAGGAAGGCGGCTGCGAGCTGAGAGTGAAGTTCAGCAGAAGCGCCGA
CGCCCCTGCCTATCAGCAGGGCCAGAACCAGCTGTACAACGAGCTGAACC
TGGGCAGACGGGAGGAATACGACGTGCTGGACAAGAGAAGAGGCCGGGAC
CCTGAGATGGGCGGCAAGCCCAGACGGAAGAACCCCCAGGAAGGCCTGTA
TAACGAACTGCAGAAAGACAAGATGGCCGAGGCCTACAGCGAGATCGGCA
TGAAGGGCGAGCGGAGAAGAGGCAAGGGCCATGACGGCCTGTACCAGGGC
CTGAGCACCGCCACCAAGGACACCTACGACGCCCTGCACATGCAGGCCCT
GCCTCCAAGATGA
Signal peptide - ligand (M505; bold) -CD8
hinge (italics)-CD8 transmembrane CD137-CD247
(SEQ ID NO: 16):
ATGGCCTTACCAGTGACCGCCTTGCTCCTGCCGCTGGCCTTGCTGCTCCA
CGCCGCCAGGCCGGGATCC-AGTAGTATTATTAGTCATTTTCGCTGGGGA
AAACCTGTTGGAGGAGGGGGGTCAGGGGGGGGTGGTGGCGCATACCCGTA
CGACGTTCCCGATTACGCT-TCCGGAACCACGACGCCAGCGCCGCGACCA
CCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACT
TCGCCTGTGATTCCGGAATCTACATCTGGGCCCCTCTGGCCGGCACCTGT
GGCGTGCTGCTGCTGTCCCTGGTCATCACCCTGTACTGCAAGCGGGGCAG
AAAGAAGCTGCTGTACATCTTCAAGCAGCCCTTCATGCGGCCTGTGCAGA
CCACACAGGAAGAGGACGGCTGTAGCTGTAGATTCCCCGAGGAAGAGGAA
GGCGGCTGCGAGCTGAGAGTGAAGTTCAGCAGAAGCGCCGACGCCCCTGC
CTATCAGCAGGGCCAGAACCAGCTGTACAACGAGCTGAACCTGGGCAGAC
GGGAGGAATACGACGTGCTGGACAAGAGAAGAGGCCGGGACCCTGAGATG
GGCGGCAAGCCCAGACGGAAGAACCCCCAGGAAGGCCTGTATAACGAACT
GCAGAAAGACAAGATGGCCGAGGCCTACAGCGAGATCGGCATGAAGGGCG
AGCGGAGAAGAGGCAAGGGCCATGACGGCCTGTACCAGGGCCTGAGCACC
GCCACCAAGGACACCTACGACGCCCTGCACATGCAGGCCCTGCCTCCAAG
ATGA
Signal peptide - ligand (M505; bold) -CD8
transmembrane CD137-CD247 (amino acids)
(SEQ ID NO: 17):
MALPVTALLLPLALLLHAARPGS-SSIISHFRWGKPVGGGGSGGGGGAYP
YDVPDYA-SGIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPRZ
Signal peptide - ligand (M505; bold) -CD8
hinge (italics)-CD8 transmembrane CD137-CD247
(amino acids) (SEQ ID NO: 18):
MALPVTALLLPLALLLHAARPGS-SSIISHFRWGKPVGGGGSGGGGGAYP
YDVPDYA-ASTTTPAPRPPTPAPTIASQPLSLRPEACRPAAGGAVHTRGL
DFACDSGIYIWAPLAGTCGVLLLSLVITLYCKRGRKKLLYIFKQPFMRPV
QTTQEEDGCSCRFPEEEEGGCELRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPRZ
Signal peptide - ligand (MSH; bold) -CD8
transmembrane CD137-CD247 (SEQ ID NO: 19):
ATGGCCTTACCAGTGACCGCCTTGCTCCTGCCGCTGGCCTTGCTGCTCCA
CGCCGCCAGGCCGGGATCC-TCCTACTCCATGGAGCACTTTAGGTGGGGA
AAACCCGTCGGGGGTGGGGGGAGTGGAGGAGGTGGTGGCGCATACCCGTA
CGACGTTCCCGATTAC-TCCGGAATCTACATCTGGGCCCCTCTGGCCGGC
ACCTGTGGCGTGCTGCTGCTGTCCCTGGTCATCACCCTGTACTGCAAGCG
GGGCAGAAAGAAGCTGCTGTACATCTTCAAGCAGCCCTTCATGCGGCCTG
TGCAGACCACACAGGAAGAGGACGGCTGTAGCTGTAGATTCCCCGAGGAA
GAGGAAGGCGGCTGCGAGCTGAGAGTGAAGTTCAGCAGAAGCGCCGACGC
CCCTGCCTATCAGCAGGGCCAGAACCAGCTGTACAACGAGCTGAACCTGG
GCAGACGGGAGGAATACGACGTGCTGGACAAGAGAAGAGGCCGGGACCCT
GAGATGGGCGGCAAGCCCAGACGGAAGAACCCCCAGGAAGGCCTGTATAA
CGAACTGCAGAAAGACAAGATGGCCGAGGCCTACAGCGAGATCGGCATGA
AGGGCGAGCGGAGAAGAGGCAAGGGCCATGACGGCCTGTACCAGGGCCTG
AGCACCGCCACCAAGGACACCTACGACGCCCTGCACATGCAGGCCCTGCC
TCCAAGATGA
Signal peptide - ligand (MSH; bold) -CD8
hinge (italics)-CD8 transmembrane CD137-CD247
(SEQ ID NO: 20):
ATGGCCTTACCAGTGACCGCCTTGCTCCTGCCGCTGGCCTTGCTGCTCCA
CGCCGCCAGGCCGGGATCC-TCCTACTCCATGGAGCACTTTAGGTGGGGA
AAACCCGTCGGGGGTGGGGGGAGTGGAGGAGGTGGTGGCGCATACCCGTA
CGACGTTCCCGATTAC-TCCGGAACCACGACGCCAGCGCCGCGACCACCA
ACACCGGCGCCCACCATCGCGTCGCAGCCCCTGTCCCTGCGCCCAGAGGC
GTGCCGGCCAGCGGCGGGGGGCGCAGTGCACACGAGGGGGCTGGACTTCG
CCTGTGATTCCGGAATCTACATCTGGGCCCCTCTGGCCGGCACCTGTGGC
GTGCTGCTGCTGTCCCTGGTCATCACCCTGTACTGCAAGCGGGGCAGAAA
GAAGCTGCTGTACATCTTCAAGCAGCCCTTCATGCGGCCTGTGCAGACCA
CACAGGAAGAGGACGGCTGTAGCTGTAGATTCCCCGAGGAAGAGGAAGGC
GGCTGCGAGCTGAGAGTGAAGTTCAGCAGAAGCGCCGACGCCCCTGCCTA
TCAGCAGGGCCAGAACCAGCTGTACAACGAGCTGAACCTGGGCAGACGGG
AGGAATACGACGTGCTGGACAAGAGAAGAGGCCGGGACCCTGAGATGGGC
GGCAAGCCCAGACGGAAGAACCCCCAGGAAGGCCTGTATAACGAACTGCA
GAAAGACAAGATGGCCGAGGCCTACAGCGAGATCGGCATGAAGGGCGAGC
GGAGAAGAGGCAAGGGCCATGACGGCCTGTACCAGGGCCTGAGCACCGCC
ACCAAGGACACCTACGACGCCCTGCACATGCAGGCCCTGCCTCCAAGATG
A
Signal peptide - ligand (MSH; bold) - CD8
transmembrane CD137-CD247 (amino acids)
(SEQ ID NO: 21):
MALPVTALLLPLALLLHAARPGS-SYSMEHFRWGKPVGGGGSGGGGGAYP
YDVPDYA-SGIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPRZ
Signal peptide - ligand (MSH; bold) -CD8
hinge (italics)-CD8 transmembrane CD137-CD247
(amino acids) (SEQ ID NO: 22):
MALPVTALLLPLALLLHAARPGS-SYSMEHFRWGKPVGGGGSGGGGGAYP
YDVPDYA-ASTTTPAPRPPTPAPTIASQPLSLRPEACRPAAGGAVHTRGL
DFACDSGIYIWAPLAGTCGVLLLSLVITLYCKRGRKKLLYIFKQPFMRPV
QTTQEEDGCSCRFPEEEEGGCELRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPRZ

The results of the experiments are now described.

### Example 1: CARs Targeting MCR Reduce Tumor Growth

The present invention relates to the discovery that chimeric receptors can be used to target MCR to treat melanoma or other tumors that express MCR.

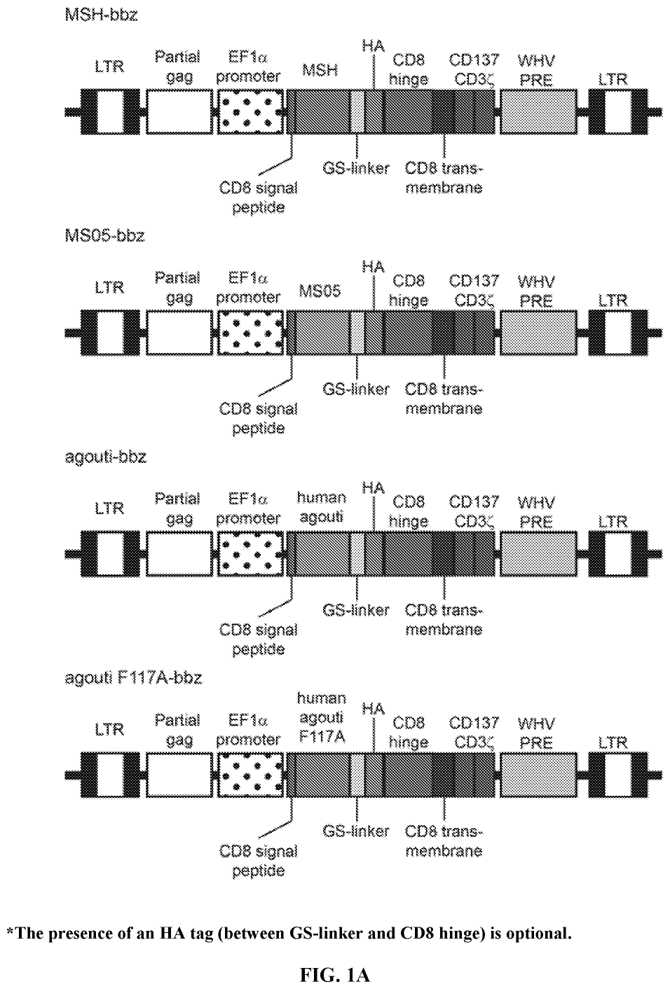

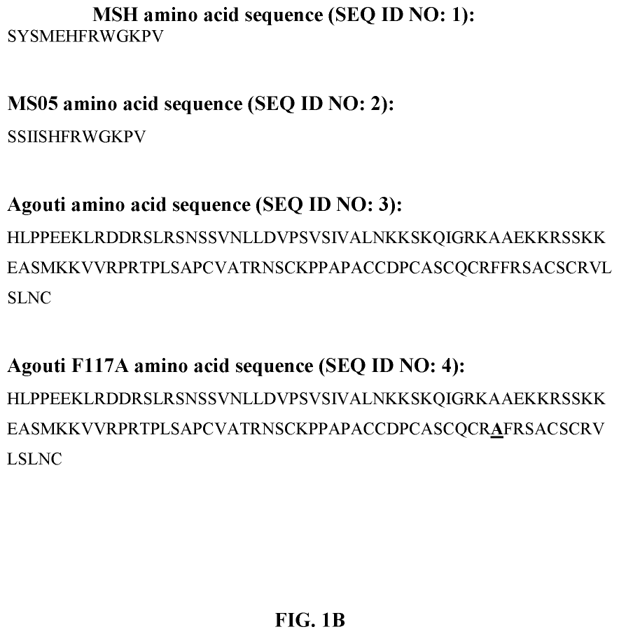

FIGS. 1A-1B illustrates the various chimeric antigen receptor constructs described in this invention (FIG. 1A) and the amino acid sequences of the ligands of interest: MSH, MS05, Agouti and Agouti F117A (FIG. 1B).

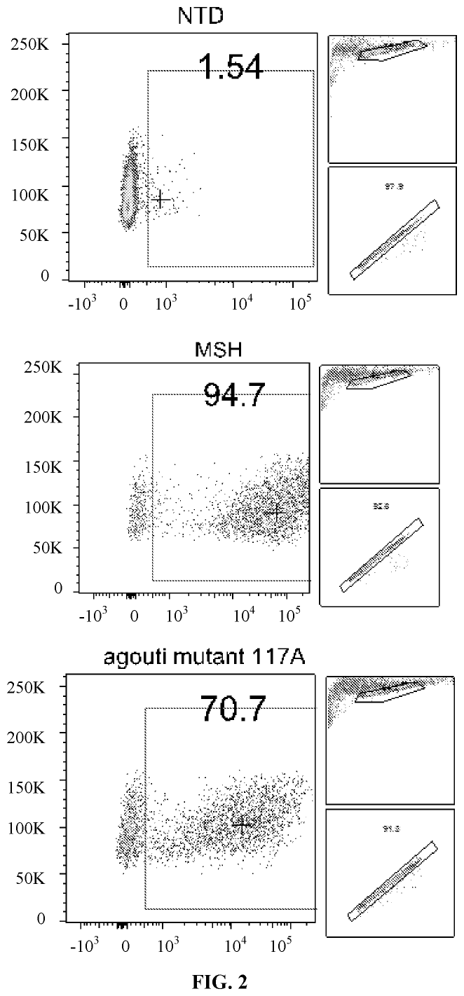

As shown in FIG. 2, a high expression of HA-tagged CAR molecules was detected on the surface of a majority of transduced human T-cells as compared to nontransduced (NTD) control cells.

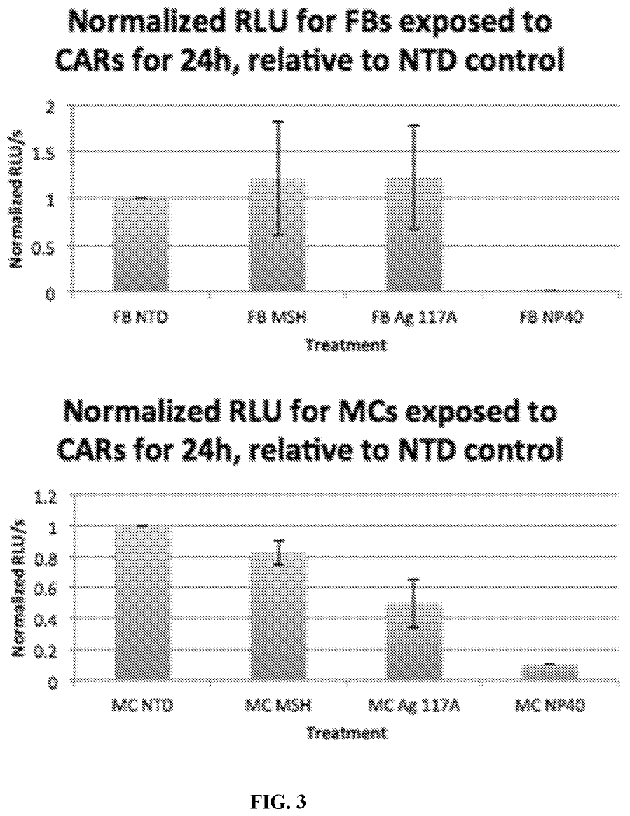

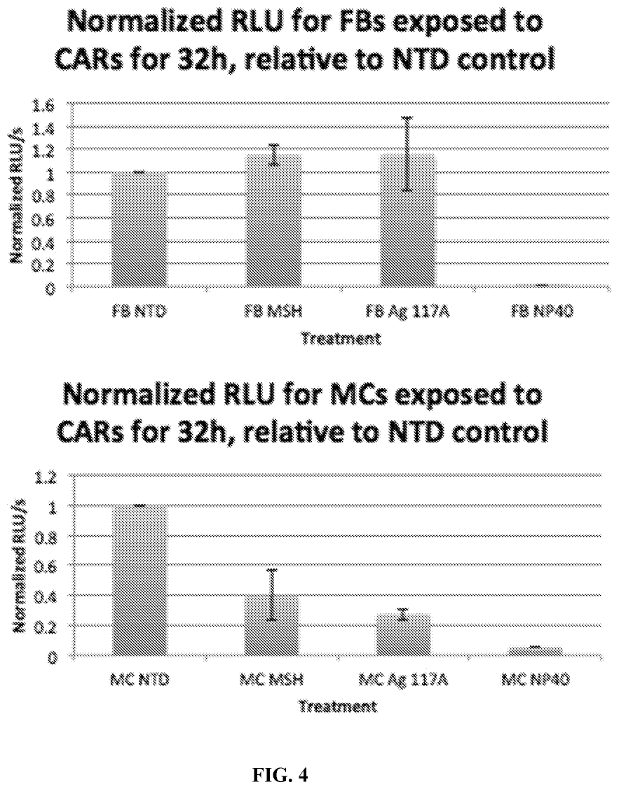

As shown in FIGS. 3A-3B, CAR-T cells with MSH or Agouti117A targeting domains selectively killed luciferase positive melanocytes at 24 hours as evidenced by the decrease in luciferase activity (measured as RLU/s) during luciferase assay (FIG. 3) and CAR-T cells with MSH or Agouti117A targeting domains display continued selective killing of melanocytes at 32 hours (FIG. 4). Fibroblasts were unaffected by the CAR-T cells as evidenced by similar luciferase activity between untreated control and CAR-T treated wells at 24 and 32h.

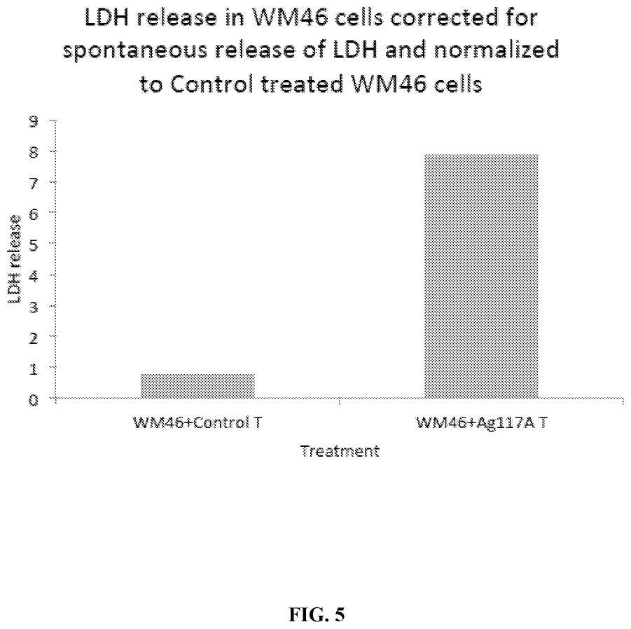

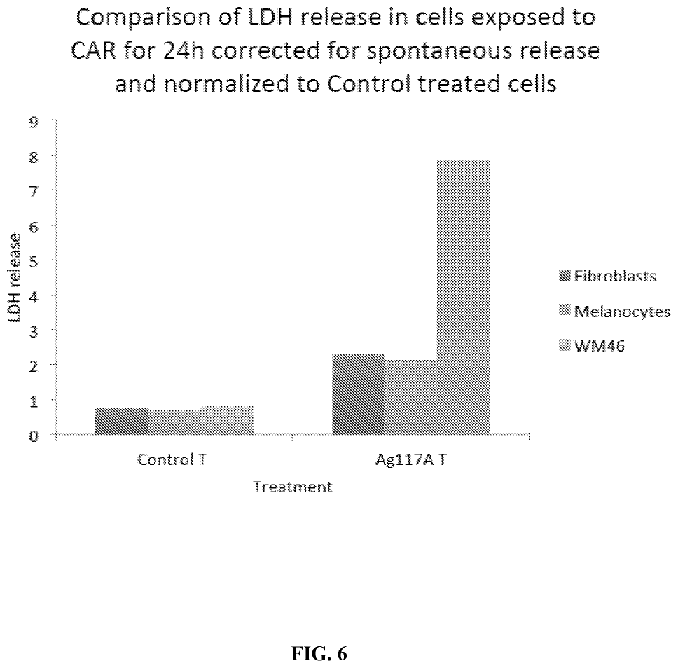

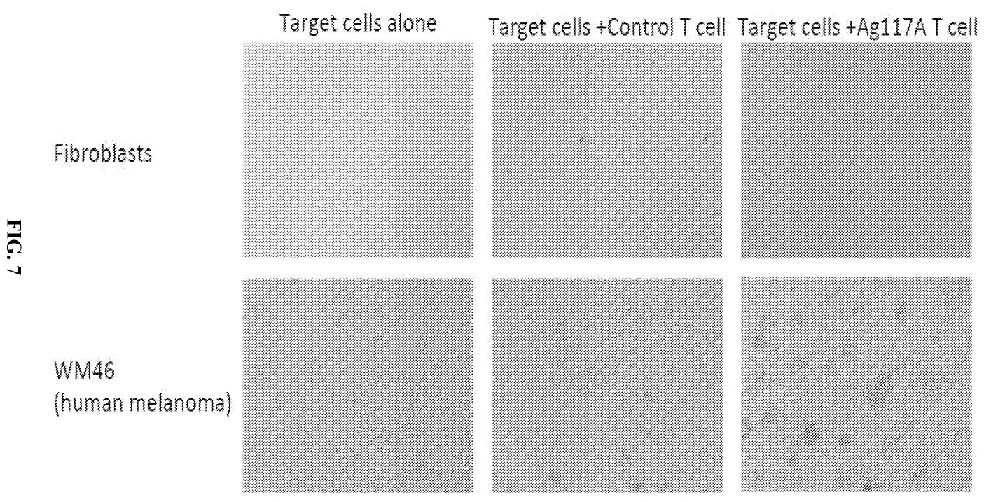

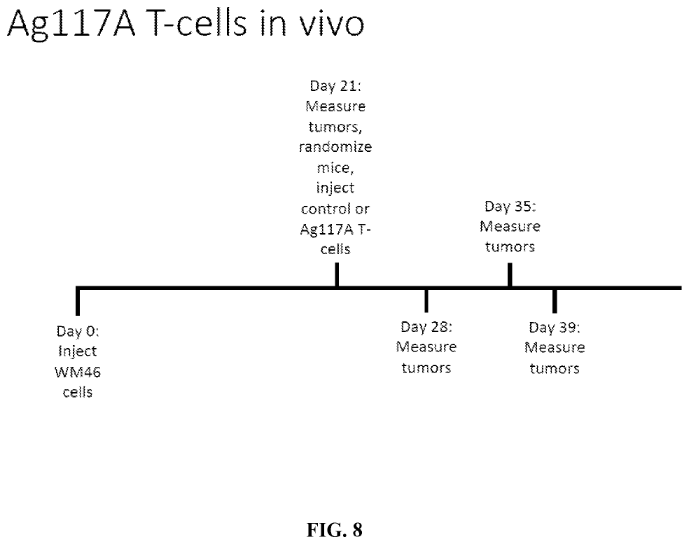

FIG. 5, FIG. 6 and FIG. 7 demonstrate that AgF117A T-cells efficiently kill melanoma cells while having little toxicity on other cell types after 24 hours of co-culture. The LDH assay, which relies on the release of lactate dehydrogenase (LDH) to detect cell death by lysis, and the cell culture images show that AgF117A T-cells effectively kill WM46 melanoma cells as evidenced by an increase in LDH release (FIG. 5, 6) and alterations in cell morphology (FIG. 7), while having little toxicity on fibroblasts and normal melanocytes.

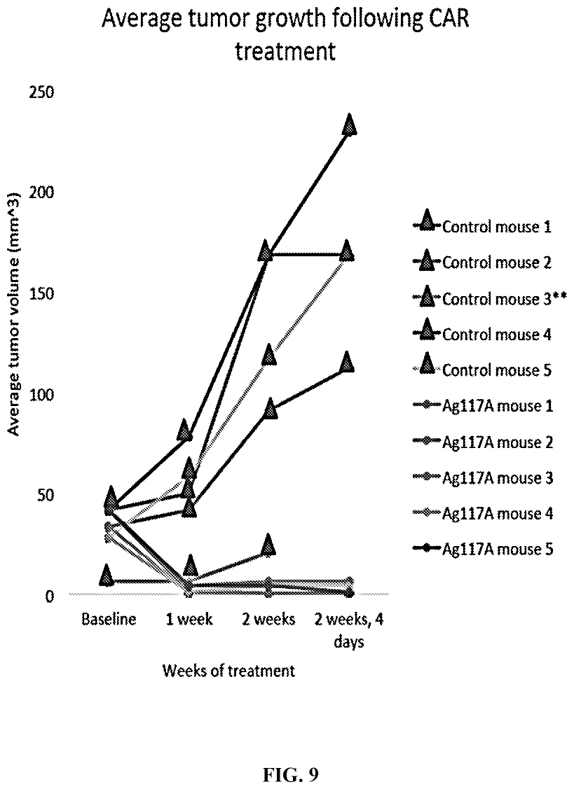

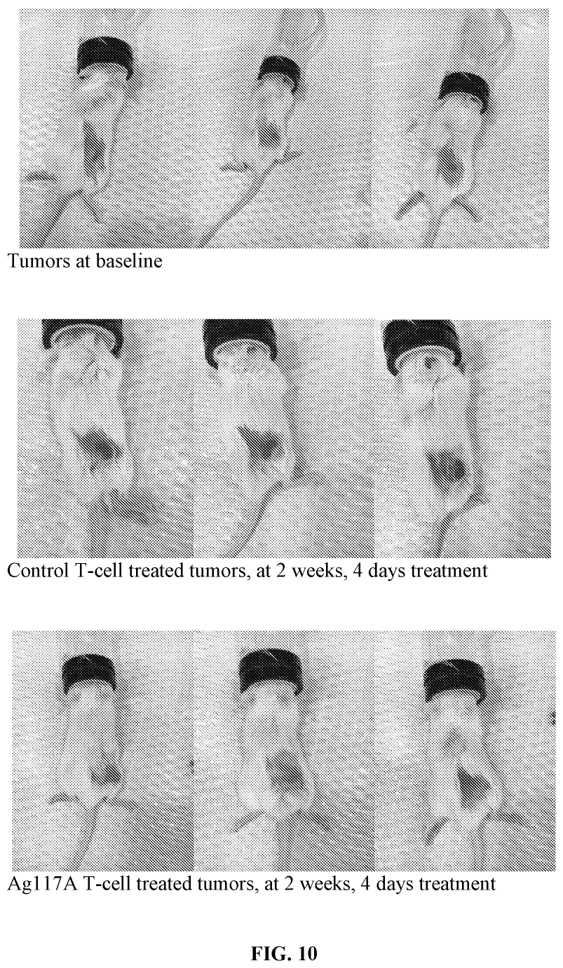

As shown in FIG. 9 and FIG. 10, following a CAR treatment in vivo, in which 10 million CAR-T cells were injected 3 weeks after the subcutaneous injection of 1 million WM46 human melanoma cells, the average tumor growth was measured biweekly. AgF117A T-cells allowed a significant regression of WM46 tumors in NSG mice.

### Example 2: CAR-T are Cytotoxic to MC1R+ SKMEL2 Cells but not MC1R− MIAPACA Cells

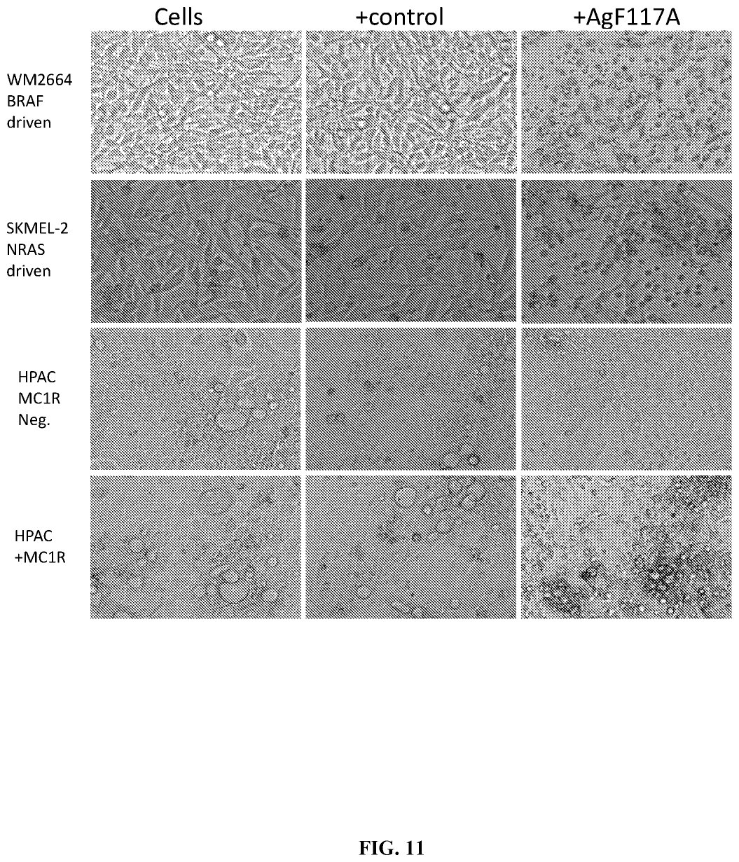

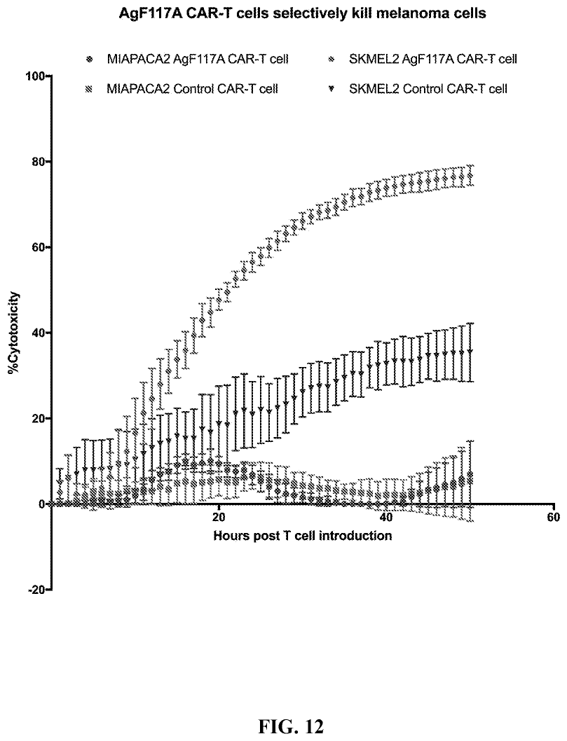

As shown in FIG. 11 and FIG. 12, MC1R negative MIAPACA2 human pancreatic cancer cells and MC1R positive SKMEL2 human melanoma cells were cocultured with control or AgF117A CAR-T cells (each condition in triplicate). Readings of adherent (target) cell death were conducted every 20 minutes. AgF117A CAR-T were cytotoxic to MC1R+ SKMEL2 cells but not MC1R− MIAPACA cells.

### Example 3: AgF117A CAR-T Cells Kill BRAF and NRAS Driven Human Melanoma

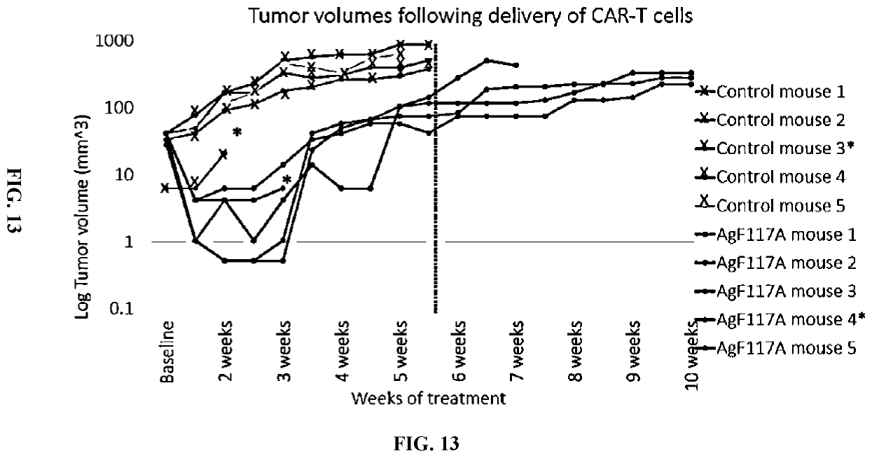

FIG. 13 shows that AgF117A CAR-T cells kill BRAF driven human melanoma. A single infusion of AgF117A T-cells caused an initial regression of WM46 tumors in NSG mice and extended survival. Tumor volumes shown on log scale. Dashed line-all control T cell treated mice euthanized due to tumor volume by 6 weeks after T cell injection, while 3/5 AgF117A CAR-T treated mice survived for 12 weeks.

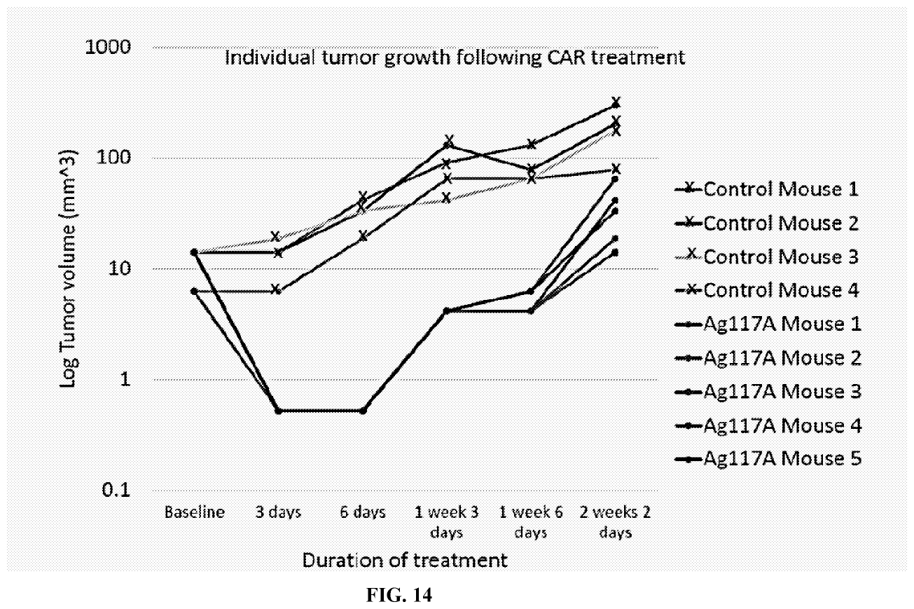

FIG. 14 shows that AgF117A CAR-T cells kill NRAS driven human melanoma.

### Example 4: Overview

This invention includes the construction and the functional testing of engineered chimeric antigen receptors (CARs) that when transduced into human T-cells direct killing of human cells expressing the surface G protein-coupled receptor MC1R including melanocytes and melanoma.

The CARs of the present invention comprise variants of the native peptide ligands for MC1R to direct selective cell killing. These ligands include αMSH (melanocyte stimulating hormone) and Agouti proteins.

MC1R is expressed on both normal melanocytes and melanoma cells, and its expression is required for cell survival. While these CAR-T cells target both normal epidermal melanocytes and melanoma cells, melanocytes are dispensable. Uveal melanocytes do not respond to MSH and therefore may not be targeted by these CARs. Loss of normal melanocytes in skin results in vitiligo, but vitiligo is now recognized as a common side effect of current immune checkpoint inhibitor therapy for melanoma, and is also common in the general population. The deficits are primarily cosmetic. Further, there is no known human disease associated with MC1R mutants other than pigmentation disorders and susceptibility to skin cancer including melanoma.

While the CAR constructs disclosed herein employ 4-1BB (CD137) and CD3 activation domains, a CD8 hinge/transmembrane domain, and HA epitope tags (FIGS. 1A-1B), many other permutations of this modular structure can be envisioned.

High-level expression of these CARs on transduced T-cells is shown via flow cytometry in FIG. 2. In some embodiment, the MCR CARs of the invention comprise a suicide gene cassette which allows for rapid killing of the CAR-T cells via systemic administration of specific small molecules.

The extracellular targeting domains described in the experiments herein include, but are not limited to, the natural αMSH sequence, and a mutant version (MS05) that displayed increased specificity for MC1R over other melanocortin receptor isoforms (MC2R-MC5R). Similarly, the Agouti F117A mutant used herein was more specific for MC1R over other melanocortin receptor isoforms.

In functional experiments, human T-cells transduced with these CAR constructs displayed specific killing of human melanocytes, with little to no killing of human fibroblasts. In the experiments shown in FIG. 3 and FIG. 4, the fibroblast and melanocyte target cells were obtained from the same human donor, indicating that the selective killing did not result from an allogeneic HLA mismatch effect.

For killing assays, target cells were first lentivirally transduced with firefly luciferase. Luciferase activity was then determined by adding luciferin to mixtures of CAR-T cells and target cells. Luciferase activity was not preserved in lysed cells as evidenced by the lack of activity in the control cells that were treated with the membrane-dissolving detergent NP-40.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated nucleic acid sequence encoding a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof.

2. The isolated nucleic acid sequence of claim 1, wherein the MCR binding domain comprises at least one MCR peptide ligand selected from the group consisting of: an alpha-melanocyte stimulating hormone (αMSH), an Agouti protein and any mutant or variant thereof.

3. The isolated nucleic acid sequence of claim 1, wherein the MCR binding domain is encoded by a nucleic acid sequence selected from the group consisting of SEQ ID NOs: 5, 7 and 9.

4. The isolated nucleic acid sequence of claim 1, wherein the transmembrane domain comprises a CD8 alpha hinge and transmembrane domain.

5. The isolated nucleic acid sequence of claim 1, wherein the signaling domain comprises a CD3 signaling domain.

6. The isolated nucleic acid sequence of claim 1, wherein the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

7. The isolated nucleic acid sequence of claim 1, wherein the CAR is encoded by a nucleic acid sequence selected from the group consisting of SEQ ID NOs: 11, 12, 15, 16, 19 and 20.

8. The isolated nucleic acid sequence of claim 1, wherein the MCR binding domain specifically binds to MCR expressed on tumor cells.

9. A vector comprising the isolated nucleic acid sequence of claim 1.

10. An isolated chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof.

11. The isolated CAR of claim 10, wherein the MCR binding domain comprises at least one MCR peptide ligand selected from the group consisting of: an alpha-melanocyte stimulating hormone (αMSH), an Agouti protein and any mutant or variant thereof.

12. The isolated CAR of claim 10, wherein the MCR binding domain comprises an amino acid sequence selected from the group consisting of SEQ ID NOs: 1-4, 6, 8 and 10.

13. The isolated CAR of claim 10, wherein the transmembrane domain comprises a CD8 alpha hinge and transmembrane domain.

14. The isolated CAR of claim 10, wherein the signaling domain comprises a CD3 signaling domain.

15. The isolated CAR of claim 10, wherein the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

16. The isolated CAR of claim 10, wherein the MCR binding domain specifically binds to MCR expressed on tumor cells.

17. The isolated CAR of claim 10, wherein the CAR comprises an amino acid sequence selected from the group consisting of SEQ ID NOs: 13, 14, 17, 18, 21 and 22.

18. A cell comprising the isolated nucleic acid sequence of claim 1 or the isolated CAR of claim 10.

19. A modified cell comprising a nucleic acid sequence encoding a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof.

20. The cell of claim 19, wherein the MCR binding domain specifically binds to MCR expressed on tumor cells.

21. The cell of claim 19, wherein the MCR binding domain binds specifically to melanoma tumors cells.

22. The cell of claim 19, wherein the cell is selected from the group consisting of a T cell, a natural killer (NK) cell, a cytotoxic T lymphocyte (CTL), a regulatory T cell and a macrophage.

23. A composition comprising the modified cell of claim 19.

24. (canceled)

25. A method for stimulating a T cell-mediated immune response to a melanocyte cell population in a subject, the method comprising administering to the subject an effective amount of a modified cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof.

26. A method of treating a subject with a cancer or a disease, disorder and condition associated with dysregulated expression of MCR, the method comprising administering to the subject a modified T cell that expresses a chimeric antigen receptor (CAR) comprising a melanocortin receptor (MCR) binding domain, a transmembrane domain, a costimulatory signaling region, and a signaling domain, wherein the MCR binding domain comprises a MCR peptide ligand or fragment thereof, a MCR antagonist or fragment thereof, or an anti-MCR agonist or fragment thereof.

27. The method of claim 26, wherein the cancer is melanoma.

28. The method of claim 26, wherein the modified T cell is autologous to the subject.

29. The method of claim 26 further comprising administering to the subject an additional agent selected from the group consisting of a chemotherapeutic agent, an anti-cell proliferation agent, an immunotherapeutic agent, an antitumor vaccine and any combination thereof.

30. The method of claim 29, wherein the modified T cell and the additional agent are co-administered to the subject.

31. The method of claim 29, wherein the additional agent is an anti-programmed cell death 1 (PD-1) antibody.

32. An isolated nucleic acid sequence encoding a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

33. The isolated nucleic acid sequence of claim 32, wherein the MCR binding domain comprises a heavy and light chain.

34. The isolated nucleic acid sequence of claim 32, wherein the MCR binding domain is selected from the group consisting of a human antibody, a humanized antibody, and a fragment thereof.

35. The isolated nucleic acid sequence of claim 34, wherein the antibody or a fragment thereof is selected from the group consisting of a Fab fragment, a F(ab′)₂ fragment, a Fv fragment, and a single chain Fv (scFv).

36. The isolated nucleic acid sequence of claim 32, wherein the MCR binding domain specifically binds to MCR expressed on tumor cells.

37. The isolated nucleic acid sequence of claim 32, wherein the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

38. A vector comprising the isolated nucleic acid sequence of claim 32.

39. An isolated chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

40. The isolated CAR of claim 39, wherein the MCR binding domain comprises a heavy and a light chain.

41. The isolated CAR of claim 39, wherein the MCR binding domain is an antibody selected from the group consisting of a human antibody, humanized antibody, and fragment thereof.

42. The isolated CAR of claim 39, wherein the MCR binding domain is selected from the group consisting of a Fab fragment, a F(ab′)₂ fragment, a Fv fragment, and a single chain Fv (scFv).

43. The isolated CAR of claim 39, wherein the MCR binding domain specifically binds to MCR expressed by tumor cells.

44. The isolated CAR of claim 39, wherein the costimulatory signaling region comprises an intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

45. A cell comprising the isolated nucleic acid sequence of claim 32.

46. A modified cell comprising a nucleic acid sequence encoding a chimeric antigen receptor (CAR) comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

47. The cell of claim 46, wherein the MCR binding domain specifically binds to MCR expressed by tumor cells.

48. The cell of claim 46, wherein the MCR binding domain specifically binds to melanoma tumor cells.

49. The cell of claim 46, wherein the cell is selected from the group consisting of a T cell, a natural killer (NK) cell, a cytotoxic T lymphocyte (CTL), a regulatory T cell and a macrophage.

50. A composition comprising the modified cell of claim 46.

51. (canceled)

52. A method for stimulating a T cell-mediated immune response to a melanocyte cell population in a subject, the method comprising administering to the subject an effective amount of a modified cell that expresses a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

53. A method of treating a subject with a cancer or a disease, disorder and condition associated with dysregulated expression of MCR, the method comprising administering to the subject a modified T cell that expresses a chimeric antigen receptor comprising a melanocortin receptor (MCR) binding domain, a transmembrane domain, a costimulatory signaling region, and a CD3 zeta signaling domain, wherein the MCR binding domain comprises an anti-MCR antibody or a fragment thereof.

54. The method of claim 53, wherein the cancer is melanoma.

55. The method of claim 53, wherein the modified T cell is autologous to the subject.

56. The method of claim 53 further comprising administering to the subject an additional agent selected from the group consisting of a chemotherapeutic agent, an anti-cell proliferation agent, an immunotherapeutic agent, an antitumor vaccine and any combination thereof.

57. The method of claim 53, wherein the modified T cell is co-administered to the subject with an additional agent.

58. The method of claim 53, wherein the modified T cell is administered to the subject with an anti-programmed cell death 1 (PD-1) antibody.

59. (canceled)


SEQUENCE LISTING (22 sequences)

SEQ ID NO: 1 - protein, 13 aa, Homo sapiens
SYSMEHFRWGKPV

SEQ ID NO: 2 - protein, 13 aa, Artificial sequence
  MS05
SSIISHFRWGKPV

SEQ ID NO: 3 - protein, 110 aa, Homo sapiens
HLPPEEKLRDDRSLRSNSSVNLLDVPSVSIVALNKKSKQIGRKAAEKKRSSKKEASMKKVVRPRTPLSAPCVATRNSCKPPAPACCDPCASCQCRFFRSACSCRVLSLNC

SEQ ID NO: 4 - protein, 110 aa, Artificial sequence; identical to SEQ ID NO: 3
  AgoutiF117A

SEQ ID NO: 5 - DNA, 387 bp, Artificial sequence
  Agouti c-terminal of signal peptide to HA-tag
cacctgccaccagaagaaaagctcagagacgatcgctcccttcgaagcaacagtagtgtgaatctcctggacgtgccctccgtgtctatcgtcgccctcaataaaaagtccaaacagatcggcagaaaggctgcagaaaagaagaggtcaagcaagaaggaggcatccatgaaaaaggtcgtcaggcctaggacgccactctcagccccctgcgtggcgactcgcaacagctgcaaaccacctgctcccgcttgctgcgatccgtgtgcctcctgtcagtgccgattttttagatcagcatgttcatgtcgggtgctgtccctcaactgtgggggtggggggagtggaggaggtggtggtgcatacccgtacgacgttcccgattac

SEQ ID NO: 6 - protein, 130 aa, Artificial sequence
  Agouti c-terminal of signal peptide to HA-tag
HLPPEEKLRDDRSLRSNSSVNLLDVPSVSIVALNKKSKQIGRKAAEKKRSSKKEASMKKVVRPRTPLSAPCVATRNSCKPPAPACCDPCASCQCRFFRSACSCRVLSLNCGGGGSGGGGGAYPYDVPDYA

SEQ ID NO: 7 - DNA, 99 bp, Artificial sequence
  MS05 c-terminal of signal peptide to HA-tag
agtagtattattagtcattttcgctggggaaaacctgttggaggaggggggtcaggggggggtggtggcgcatacccgtacgacgttcccgattacgct

SEQ ID NO: 8 - protein, 33 aa, Artificial sequence
  MS05 c-terminal of signal peptide to HA-tag
SSIISHFRWGKPVGGGGSGGGGGAYPYDVPDYA

SEQ ID NO: 9 - DNA, 96 bp, Artificial sequence
  MSH c-terminal of signal peptide to HA-tag
tcctactccatggagcactttaggtggggaaaacccgtcgggggtggggggagtggaggaggtggtggcgcatacccgtacgacgttcccgattac

SEQ ID NO: 10 - protein, 33 aa, Artificial sequence
  MSH c-terminal of signal peptide to HA-tag
SYSMEHFRWGKPVGGGGSGGGGGAYPYDVPDYA

SEQ ID NO: 11 - DNA, 999 bp, Artificial sequence
  Signal peptide - ligand (Agouti) -CD8 transmembrane CD137-CD247
atggccttaccagtgaccgccttgctcctgccgctggccttgctgctccacgccgccaggccgggatcccacctgccaccagaagaaaagctcagagacgatcgctcccttcgaagcaacagtagtgtgaatctcctggacgtgccctccgtgtctatcgtcgccctcaataaaaagtccaaacagatcggcagaaaggctgcagaaaagaagaggtcaagcaagaaggaggcatccatgaaaaaggtcgtcaggcctaggacgccactctcagccccctgcgtggcgactcgcaacagctgcaaaccacctgctcccgcttgctgcgatccgtgtgcctcctgtcagtgccgattttttagatcagcatgttcatgtcgggtgctgtccctcaactgtgggggtggggggagtggaggaggtggtggtgcatacccgtacgacgttcccgattactccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 12 - DNA, 1140 bp, Artificial sequence
  Signal peptide - ligand (Agouti) -CD8 hinge-CD8 transmembrane CD137-CD247
atggccttaccagtgaccgccttgctcctgccgctggccttgctgctccacgccgccaggccgggatcccacctgccaccagaagaaaagctcagagacgatcgctcccttcgaagcaacagtagtgtgaatctcctggacgtgccctccgtgtctatcgtcgccctcaataaaaagtccaaacagatcggcagaaaggctgcagaaaagaagaggtcaagcaagaaggaggcatccatgaaaaaggtcgtcaggcctaggacgccactctcagccccctgcgtggcgactcgcaacagctgcaaaccacctgctcccgcttgctgcgatccgtgtgcctcctgtcagtgccgattttttagatcagcatgttcatgtcgggtgctgtccctcaactgtgggggtggggggagtggaggaggtggtggtgcatacccgtacgacgttcccgattactccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 13 - protein, 334 aa, Artificial sequence
  Signal peptide - ligand (Agouti) -CD8 transmembrane CD137-CD247 amino acids
MALPVTALLLPLALLLHAARPGSHLPPEEKLRDDRSLRSNSSVNLLDVPSVSIVALNKKSKQIGRKAAEKKRSSKKEASMKKVVRPRTPLSAPCVATRNSCKPPAPACCDPCASCQCRFFRSACSCRVLSLNCGGGGSGGGGGAYPYDVPDYASGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ

SEQ ID NO: 14 - protein, 381 aa, Artificial sequence
  Signal peptide - ligand (Agouti) -CD8 hinge-CD8 transmembrane CD137-CD247 (amino acids)
MALPVTALLLPLALLLHAARPGSHLPPEEKLRDDRSLRSNSSVNLLDVPSVSIVALNKKSKQIGRKAAEKKRSSKKEASMKKVVRPRTPLSAPCVATRNSCKPPAPACCDPCASCQCRFFRSACSCRVLSLNCGGGGSGGGGGAYPYDVPDYAASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ

SEQ ID NO: 15 - DNA, 711 bp, Artificial sequence
  Signal peptide - ligand (MS05) -CD8 transmembrane CD137-CD247
atggccttaccagtgaccgccttgctcctgccgctggccttgctgctccacgccgccaggccgggatccagtagtattattagtcattttcgctggggaaaacctgttggaggaggggggtcaggggggggtggtggcgcatacccgtacgacgttcccgattacgcttccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 16 - DNA, 852 bp, Artificial sequence
  Signal peptide - ligand (MS05) -CD8 hinge-CD8 transmembrane CD137-CD247
atggccttaccagtgaccgccttgctcctgccgctggccttgctgctccacgccgccaggccgggatccagtagtattattagtcattttcgctggggaaaacctgttggaggaggggggtcaggggggggtggtggcgcatacccgtacgacgttcccgattacgcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 17 - protein, 237 aa, Artificial sequence
  Signal peptide - ligand (MS05) -CD8 transmembrane CD137-CD247 (amino acids)
MALPVTALLLPLALLLHAARPGSSSIISHFRWGKPVGGGGSGGGGGAYPYDVPDYASGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ

SEQ ID NO: 18 - protein, 284 aa, Artificial sequence
  Signal peptide - ligand (MS05) -CD8 hinge-CD8 transmembrane CD137-CD247 (amino acids)
MALPVTALLLPLALLLHAARPGSSSIISHFRWGKPVGGGGSGGGGGAYPYDVPDYAASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ

SEQ ID NO: 19 - DNA, 708 bp, Artificial sequence
  Signal peptide - ligand (MSH) -CD8 transmembrane CD137-CD247
atggccttaccagtgaccgccttgctcctgccgctggccttgctgctccacgccgccaggccgggatcctcctactccatggagcactttaggtggggaaaacccgtcgggggtggggggagtggaggaggtggtggcgcatacccgtacgacgttcccgattactccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 20 - DNA, 849 bp, Artificial sequence
  Signal peptide - ligand (MSH) -CD8 hinge-CD8 transmembrane CD137-CD247
atggccttaccagtgaccgccttgctcctgccgctggccttgctgctccacgccgccaggccgggatcctcctactccatggagcactttaggtggggaaaacccgtcgggggtggggggagtggaggaggtggtggcgcatacccgtacgacgttcccgattactccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 21 - protein, 237 aa, Artificial sequence
  Signal peptide - ligand (MSH) - CD8 transmembrane CD137-CD247 (amino acids)
MALPVTALLLPLALLLHAARPGSSYSMEHFRWGKPVGGGGSGGGGGAYPYDVPDYASGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ

SEQ ID NO: 22 - protein, 284 aa, Artificial sequence
  Signal peptide - ligand (MSH) -CD8 hinge-CD8 transmembrane CD137-CD247 (amino acids)
MALPVTALLLPLALLLHAARPGSSYSMEHFRWGKPVGGGGSGGGGGAYPYDVPDYAASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRZ